(19) United States
(12) Patent Application Publication
(10) Pub. No.: US 2007/0027383 A1
(43) Pub. Date: Feb. 1, 2007

(54) Patches, systems, and methods for non-invasive glucose measurement

(76) Inventors: Thomas A. Peyser, Menlo Park, CA (US); Russell O. Potts, San Francisco, CA (US); Herbert L. Berman, Los Altos Hills, CA (US); James W. Moyer, San Francisco, CA (US); Mikhail A. Kouchnir, Sunnyvale, CA (US); Robert N. Blair, San Jose, CA (US)

Correspondence Address: MORRISON & FOERSTER LLP, 755 PAGE MILL RD, PALO ALTO, CA, 94304-1018, US

(21) Appl. No.: 11/451,738

(22) Filed: Jun. 12, 2006

Related U.S. Application Data
(63) Continuation-in-part of application No. 11/159,587, filed on Jun. 22, 2005.
(60) Provisional application No. 60/585,414, filed on Jul. 1, 2004.

(51) Int. Cl. A61B 5/05; A61B 5/00

(52) U.S. Cl. 600/347; 600/365; 600/346; 600/362

(57) ABSTRACT

Described here are patches, systems, and methods for measuring glucose. In general, the patches comprise a microfluidic collection layer and a detector, and the systems comprise a patch and a measurement device. Some methods for measuring glucose comprise cleaning the skin surface, collecting sweat from the skin surface using a microfluidic collection device, and measuring the collected glucose. Other methods comprise cleaning the skin surface, collecting sweat in a patch comprising a microfludic collection layer, and measuring glucose collected in the patch. Still other methods comprise cleaning the skin surface, collecting a first sweat sample from the skin surface in a patch comprising a microfludic collection layer and a detector layer, transferring the first sweat sample from the collection layer to the detector layer, measuring glucose in the first sweat sample, and repeating the collection, transferring, and measuring steps at least once.

### CROSS REFERENCE TO RELATED APPLICATIONS

This application is a continuation-in-part of U.S. Ser. No. 11/159,587, filed Jun. 22, 2005, which claims priority to U.S. Ser. No. 60/585,414, filed on Jul. 1, 2004, both of which are hereby incorporated by reference in their entirety.

### FIELD

The devices, methods, and systems described here are in the field of non-invasive glucose measurement, and more specifically, non-invasive measurement of nanogram quantities of glucose, which have come to the skin surface via sweat.

### BACKGROUND

The American Diabetes Association reports that approximately 6% of the population in the United States, a group of 16 million people, has diabetes, and that this number is growing at a rate of 12-15% per annum. The Association further reports that diabetes is the seventh leading cause of death in the United States, contributing to nearly 200,000 deaths per year. Diabetes is a life-threatening disease with broad complications, which include blindness, kidney disease, nerve disease, heart disease, amputation and stroke. Diabetes is believed to be the leading cause of new cases of blindness in individuals aging between 20 and 74; approximately 12,000 people per year lose their sight because of diabetes. Diabetes is also the leading cause of end-stage renal disease, accounting for nearly 40% of new cases. Nearly 60-70% of people with diabetes have mild to severe forms of diabetic nerve damage which, in severe forms, can lead to lower limb amputations. People with diabetes are 2-4 times more likely to have heart disease and to suffer strokes.

Diabetes results from the inability of the body to produce or properly use insulin, a hormone needed to convert sugar, starches, and the like into energy. Although the cause of diabetes is not completely understood, genetics, environmental factors, and viral causes have been partially identified.

There are two major types of diabetes: Type 1 and Type 2. Type 1 diabetes (also known as juvenile diabetes) is caused by an autoimmune process destroying the beta cells that secrete insulin in the pancreas. Type 1 diabetes most often occurs in young adults and children. People with Type 1 diabetes must take daily insulin injections to stay alive.

Type 2 diabetes is a metabolic disorder resulting from the body's inability to make enough, or properly to use, insulin. Type 2 diabetes is more common, accounting for 90-95% of diabetes. In the United States, Type 2 diabetes is nearing epidemic proportions, principally due to an increased number of older Americans and a greater prevalence of obesity and sedentary lifestyles.

Insulin, in simple terms, is the hormone that allows glucose to enter cells and feed them. In diabetics, glucose cannot enter the cells, so glucose builds up in the blood to toxic levels.

Diabetics having Type 1 diabetes are typically required to self-administer insulin using, e.g., a syringe or a pen with needle and cartridge. Continuous subcutaneous insulin infusion via external or implanted pumps is also available. Diabetics having Type 2 diabetes are typically treated with changes in diet and exercise, as well as with oral medications. Many Type 2 diabetics become insulin-dependent at later stages of the disease. Diabetics using insulin to help regulate their blood sugar levels are at an increased risk for medically-dangerous episodes of low blood sugar due to errors in insulin administration, or unanticipated changes in insulin absorption.

It is highly recommended by the medical profession that insulin-using patients practice self-monitoring of blood glucose (“SMBG”). Based upon the level of glucose in the blood, individuals may make insulin dosage adjustments before injection. Adjustments are necessary since blood glucose levels vary day to day for a variety of reasons, e.g., exercise, stress, rates of food absorption, types of food, hormonal changes (pregnancy, puberty, etc.) and the like. Despite the importance of SMBG, several studies have found that the proportion of individuals who self-monitor at least once a day significantly declines with age. This decrease is likely due simply to the fact that the typical, most widely used, method of SMBG involves obtaining blood from a capillary finger stick. Many patients consider obtaining blood to be significantly more painful than the self-administration of insulin.

Non- or minimally-invasive techniques are being investigated, some of which are beginning to focus on the measurement of glucose on the skin surface or in interstitial fluid. For example, U.S. Pat. No. 4,821,733 to Peck describes a process to detect an analyte that has come to the skin surface via diffusion. Specifically, Peck teaches a transdermal detection system for the detection of an analyte that migrates to the skin surface of a subject by diffusion in the absence of a liquid transport medium, such as sweat. As will be described in more detail below, because the process of passive diffusion of an analyte to the skin surface takes an unreasonably long period of time (e.g., a few hours to several days), Peck does not provide a practical non-invasive glucose monitoring solution.

Similarly, U.S. Pat. No. 6,503,198 to Aronowitz et al. (“Aronowitz”) describes a transdermal system for analyte extraction from interstitial fluid. Specifically, Aronowitz teaches patches containing wet and dry chemistry components. The wet component is used to form a gel layer for the extraction and liquid bridge transfer of the analyte from the biological fluid to the dry chemistry component. The dry chemistry component is used to quantitatively or qualitatively measure the analyte. One disadvantage of the system described in Aronowitz is the effect of a wet chemistry interface in providing a liquid phase environment on the skin in which different sources of glucose could be irreversibly mixed with one another. A liquid phase contact with the skin surface could make it impossible to distinguish between glucose on the skin surface originating from many day old epidermal debris, glucose on the skin surface originating from many hours old transdermal diffusion, and finally, glucose on the skin from the more timely output of the eccrine sweat gland.

Others have investigated glucose measurement in sweat; however, they have failed to demonstrate a correlation between blood glucose levels and sweat glucose levels, and have similarly failed to establish or demonstrate that only glucose coming from sweat is being measured. For example, U.S. Pat. No. 5,140,985 to Schroeder et al. (“Schroeder”) describes a non-invasive glucose monitoring unit, which uses a wick to absorb the sweat and electrochemistry to make glucose measurements. Schroeder relies on an article by T. C. Boysen, Shigeree Yanagaun, Fusaho Sato and Uingo Sato published in 1984 in the Journal of Applied Psychology to establish the correlation between blood glucose and sweat glucose levels, but quantitative analysis of the data provided therein demonstrates that the blood glucose and sweat glucose levels of the two subjects described there cannot be correlated (yielding correlation coefficients of approximately 0.666 and 0.217 respectively). Additional methods must be used, beyond those cited in the paper by Boysen et al., to isolate the glucose in sweat from other sources of glucose on the skin.

Similarly, U.S. Pat. No. 5,036,861 to Sembrowich et al. (“Sembrowich”) describes glucose monitoring technology based on analyzing glucose on the skin surface from a localized, modified sweat response. In a like manner, U.S. Pat. No. 5,638,815 to Schoendorfer (“Schoendorfer”) describes a dermal patch to be worn on the skin for increasing the concentration of an analyte expressed through the skin in perspiration, to a conveniently measurable level. However, similar to Schroeder, Sembrowich and Schoendorfer each fail to teach or describe methods or steps for isolating or distinguishing the glucose in sweat from other confounding sources of glucose found on the skin surface.

Because disorders such as diabetes are chronic and have ongoing effects, there is also a need for effective and economical methods of monitoring a subject's glucose at multiple time points, and for devices capable of executing these methods.

### BRIEF SUMMARY

Described here are patches, systems, and methods for monitoring glucose. In general, the patches comprise a microfluidic collection layer and a detector. The microfluidic collection layer may have a number of different configurations. For example, the microfluidic collection layer may be serpentine in nature, or may comprise concentric microfluidic channels. The microfluidic collection layer may also be composed of a series of micro-channels that collect sweat by capillary action in a “wicking” action. Similarly, the detector may be any suitable detector. For example, the detector may be an electrochemical detector (e.g., glucose oxidase). The detector may be substantially immobilized within the patch, or may be in solution. In some variations, the detector is in a detector layer, which may or may not be in fluid communication with the collection layer.

The patch may also comprise a sweat-permeable membrane configured to act as a barrier to epidermal contaminants and glucose brought to the skin surface via diffusion. The sweat-permeable membrane may be made of a material that is generally occlusive, but allows sweat to pass therethrough or may be made of a liquid polymer that cures when exposed to oxygen and leaves openings over the sweat gland pores. Other alternative sweat-permeable membranes may also be used.

The patch may also comprise an adhesive or an adhesive layer, for example, to help adhere the patch to the skin surface. Similarly, the patch may also comprise a mechanism for inducing sweat. The mechanism may be mechanical (e.g., an occlusive backing layer, vacuum, etc.), chemical (e.g., sweat inducers such as pilocarpine with or without a penetration enhancer or iontophoresis), or thermal (e.g., a heater, etc.). In some variations, the mechanism for inducing sweat is in the collection layer.

Also described here are glucose monitoring systems. In general the glucose monitoring system comprises a patch configured to collect a nanogram quantity of glucose in sweat, where the patch comprises a microfluidic collection layer and a detector and a measurement device configured to measure the nanogram quantity of glucose. As with the patches described above, the patches of the system may also comprise a sweat-permeable membrane configured to act as a barrier to epidermal contaminants and glucose brought to the skin surface via diffusion, an adhesive or an adhesive layer, and a mechanism for inducing sweat. That is, any of the patch variations described just above may be used with the patch described here as part of the glucose monitoring systems.

The systems described here may also include a pump. The pump may be an active pump (e.g., positional displacement pumps such as gear or peristaltic pumps, piezoelectric pumps, membrane pumps, etc.) or a passive pump (e.g., thermal pumps, osmotic pumps, a preloaded pressure bolus, etc.). The systems may also comprise a buffer. The buffer may be at physiological pH and be isotonic. In some variations, the buffer is Phosphate Buffered Saline or “PBS.”

The measurement devices of the systems described here may also comprise a display, a process, computer executable code for executing a calibration algorithm, and a measurement mechanism for measuring glucose collected in the patch. In some variations, the measurement device is placed on the patch for extended periods of time (e.g., the measurement device is worn by the user), or repeatedly applied to the patch at pre-determined time intervals. The system may also comprise a device for measuring relative humidity, which may or may not be part of the measurement device.

As noted above, methods for measuring glucose on the skin surface are also provided here. Some methods generally comprise cleaning the skin surface with a glucose solvent, collecting sweat from the skin surface using a microfluidic collection device, and measuring the collected glucose. The method may also include a step of inducing sweat prior to collecting the sweat from the skin surface. The step of inducing sweat may comprise inducing sweat mechanically (e.g., by using an occlusive backing layer, a vacuum, etc.), chemically (e.g., by administering sweat inducing agents such as pilocarpine with or without a penetration enhancer or iontophoresis), or thermally (e.g., by applying a heater, or initiating an exothermic chemical reaction, etc.). In some variations, measuring comprises measuring nanogram quantities of glucose.

Other methods for measuring glucose on the skin surface comprise cleaning the skin surface with a glucose solvent, collecting sweat from the skin surface in a patch comprising a microfludic collection layer, and measuring glucose collected in the patch. Again, any of the patch variations described above may be used with the patch described here as part of the methods. In some variations, collecting sweat comprises collecting sweat in a microfludic collection layer containing a buffer.

The method may also include pumping a buffer into the microfluidic collection layer (e.g., after collecting the sweat). In these variations, the patch typically has a collection layer and a detector layer, which are in fluid communication with each other. In this way, the sweat sample may be moved from the collection layer to the detector layer for glucose detection and measurement. Of course, it should be understood that any of the steps of the method may be repeated (e.g., collecting the sweat and measuring the glucose).

Still other methods for measuring glucose on a skin surface comprise cleaning the skin surface with a glucose solvent, collecting a first sweat sample from the skin surface in a patch comprising a microfludic collection layer and a detector layer, transferring the first sweat sample from the collection layer to the detector layer, measuring glucose in the first sweat sample, and repeating the collection, transferring, and measuring steps at least once.

The step of collecting the first sweat sample may comprise collecting the first sweat sample in a microfludic collection layer containing a buffer or may comprise collecting the first sweat sample in a microfluidic collection layer devoid of a buffer. Similarly, the step of transferring the first sweat sample from the collection layer to the detector layer may comprise pumping a buffer into the microfluidic collection layer or may comprise applying pressure (e.g., gas pressure, liquid pressure, or mechanical pressure) within the microfludic collection layer. For example, in some variations, pressure is used to transfer the sweat sample and pressure is applied with pressurized saline. Other variations for transferring the sweat sample may also be used.

The steps may be repeated after a predetermined period of time, e.g., less than about 60 minutes, less than about 30 minutes, less than about 20 minutes, less than about 10 minutes, less than about 5 minutes, etc. Similarly, the steps may be repeated for a predetermined period of time, e.g., about 1 hour, about 2 hours, about 3 hours, about 4 hours, about 5 hours, about 6 hours, etc. These periods of time may be set automatically, or may be set manually.

The methods described here may also include the step of inducing a sweat prior to collecting a first sweat sample. The step of inducing sweat may comprise inducing sweat mechanically (e.g., by using an occlusive backing layer, a vacuum, etc.), chemically (e.g., by administering sweat inducing agents such as pilocarpine with or without a penetration enhancer or iontophoresis), or thermally (e.g., by applying a heater, or initiating an exothermic chemical reaction, etc.).

### BRIEF DESCRIPTION OF THE DRAWINGS

FIG. 1 provides a schematic of glucose transport mechanisms from the blood to the skin.

FIGS. 2A and 2B provide cross-sectional views of illustrative patches described herein.

FIGS. 3A, 3B, 3C and 3D provide illustrative microfluidic collection layers as described herein.

FIG. 4 shows the effect of thermal stimulation on the sweat response over time.

FIGS. 5A-5G show illustrative variations of how a fixed volume reservoir may be used with the patches described herein.

FIG. 6 provides a schematic representation of an exemplary glucose monitoring system that may be used herein.

FIG. 7 provides a flow chart of one exemplary method for measuring glucose from the skin surface as described herein.

FIG. 8 shows the results of glucose measurements with and without the use of a sweat-permeable membrane.

FIG. 9 demonstrates a normalized correlation between blood glucose and sweat glucose when a sweat-permeable membrane is used.

FIG. 10 is a plot of the ratio of sweat flux to glucose flux with and without a sweat-permeable membrane.

FIG. 11 is a plot demonstrating the sweat and blood glucose levels in a subject having falling glucose levels.

FIGS. 12A and 12B provide regression plots for the data plotted in FIG. 11.

FIG. 13 is a plot demonstrating the sweat and blood glucose levels in a subject having rising glucose levels.

FIGS. 14A and 14B provide regression plots for the data plotted in FIG. 13.

### DETAILED DESCRIPTION

Described here are patches, systems, and methods for monitoring glucose. In general, the patches comprise a microfluidic collection layer and a detector. Similarly, the glucose monitoring systems described herein comprise a patch configured to collect a nanogram quantity of glucose in sweat, where the patch comprises a microfluidic collection layer and a detector and a measurement device configured to measure the nanogram quantity of glucose. Lastly, methods for monitoring glucose are also described here. In some variations, the methods generally comprise cleaning the skin surface with a glucose solvent, collecting sweat from the skin surface using a microfluidic collection device, and measuring the collected glucose. These methods may also include a step of inducing sweat prior to collecting the sweat from the skin surface. Other methods for measuring glucose on the skin surface comprise cleaning the skin surface with a glucose solvent, collecting sweat from the skin surface in a patch comprising a microfludic collection layer; and

measuring glucose collected in the patch. Still other methods for measuring glucose on a skin surface comprise cleaning the skin surface with a glucose solvent, collecting a first sweat sample from the skin surface in a patch comprising a microfludic collection layer and a detector layer, transferring the first sweat sample from the collection layer to the detector layer, measuring glucose in the first sweat sample, and repeating the collection, transferring, and measuring steps at least once. The methods, systems, and devices described herein provide a way to measure glucose brought to the skin via sweat, which is correlatable to blood glucose as will be described in more detail below. It should be understood that when reference is made to the term “skin” herein throughout, that term it is meant to include, not only the outermost skin surface, but also, the entire stratum corneum. The patches, systems and methods will be described in more detail below.

Patches

In general, the patches comprise a microfluidic collection layer and a detector. The microfluidic collection layer may have a number of different configurations. For example, the microfluidic collection layer may be serpentine in nature, or may comprise concentric microfluidic channels. Similarly, the detector may be any suitable detector. For example, the detector may be an electrochemical detector (e.g., glucose oxidase). The detector may be substantially immobilized within the patch, or may be in solution. In some variations, the detector is in a detector layer, which may or may not be in fluid communication with the collection layer.

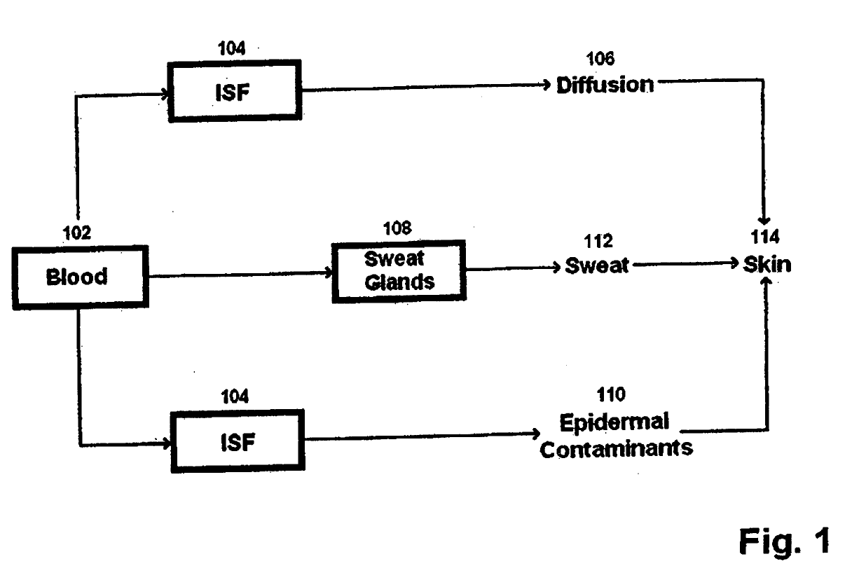

The patch may also comprise a sweat-permeable membrane configured to act as a barrier to epidermal contaminants and glucose brought to the skin surface via diffusion. For example, as shown in FIG. 1, there are different routes by which the glucose in blood migrates to the skin over time. As shown there, the glucose in blood (102) passes to the interstitial fluid (104), or to sweat glands (108). After a period of time, the glucose levels in blood (102) and glucose levels in the interstitial fluid (104) reach equilibrium. In healthy subjects, this period of time is typically on the order of five to ten minutes. This relatively short time delay for equilibrium achievement between blood glucose and interstitial fluid glucose levels has made interstitial fluid the focus of many efforts to develop continuous glucose monitoring technology.

Glucose derived from the interstitial fluid (104) is also transported by diffusion (106) through the stratum corneum to the skin surface. However, the relative impermeability of the stratum corneum, or alternatively, the high quality of the barrier function of intact stratum corneum tissue, results in significant time delays for the passage across the stratum corneum by transdermal diffusion. The glucose delivered to the skin surface by transdermal diffusion lags behind blood glucose by many hours making it unsuitable for medical diagnostic uses.

Glucose may also arrive on the skin surface via the process of stratum corneum desquamation resulting in epidermal contaminants (110), and the like. For example, epidermal glucose results from the specific enzymatic cleavage of certain lipids. This produces free glucose, a source of energy for the upper layers of the epidermis which are avascular and therefore not perfused with blood. This free glucose is not representative of the corresponding blood glucose, or of the interstitial glucose values.

The sweat gland (108) may be considered a shunt that traverses the stratum corneum and allows rapid mass transport of material through an otherwise relatively impermeable barrier. Glucose from the interstitial fluid is the primary source of energy for the work-or-pump function of the eccrine sweat glands (108). The sweat secreted by the eccrine sweat gland contains a fraction of glucose from the blood (102), which erupts from the skin through tiny pores or orifices on the skin surface. We have discovered that a fraction of the secreted sweat may be re-absorbed by the stratum corneum. The amount of sweat, and consequently, the amount of glucose, back-absorbed into the stratum corneum depends on the hydration state of the skin and varies throughout the day. In addition, the water in sweat may extract glucose from the stratum corneum. Thus, without blocking the back transfer of glucose between sweat and the stratum corneum, it may be difficult to develop an instrument that could correlate the glucose on the skin with that in the blood.

Cunningham and Young measured the glucose content in the stratum corneum using a variety of methods including serial tape stripping and aqueous extraction, and found approximately 10 nanograms per square centimeter per micron of depth of stratum corneum. See Cunningham, D. D. and Young, D. F., “Measurements of Glucose on the Skin Surface, in Stratum Corneum and in Transcutaneous Extracts: Implications for Physiological Sampling”, Clin. Chem. Lab Med, 41, 1224-1228, 2003. In their experiments in collecting and harvesting glucose from the skin surface, Cunningham and Young found that the stratum corneum was the source of epidermal contaminants on the skin surface, and that these contaminants were not correlatable to blood glucose.

The glucose from epidermal contaminants typically reflects glucose abundance in the tissue anywhere from days to weeks prior to its appearance during desquamation (because epidermal turnover occurs approximately every 28 days). See, e.g., Rao, G., Guy, R. H., Glikfeld, P., LaCourse, W. R., Leung, L. Tamada, J., Potts, R. O., Azimi, N. “Reverse iontophoresis: noninvasive glucose monitoring in vivo in humans,” Pharm Res, 12, 1869-1873 (1995). In a like manner, it is unlikely that the glucose brought to the skin surface via diffusion (106) can be correlated to blood glucose. In addition, because the glucose has to traverse the tortuous path of the skin layers to reach the surface, the glucose brought to the skin surface via diffusion often results in a lag time (e.g., in the range of a few hours to days), which is undesirable for purposes of glucose monitoring.

The sweat-permeable membrane may also aid in preventing or minimizing the re-absorption of glucose that has been brought to the skin surface via sweat, in the outer layer of the stratum corneum. In general, the sweat-permeable membrane may comprise any material that allows sweat to pass therethrough, is non-toxic, and prevents glucose brought to the skin surface via diffusion or epidermal contamination from entering the collection layer. As mentioned just above, it may also prevent reabsorption of the sweat into the skin. For example, the sweat-permeable membrane may be made of a hydrophobic coating or a porous hydrophobic film. The film should be thick enough to coat the skin, but thin enough to allow sweat to pass therethrough. Suitable examples of hydrophobic materials include petrolatum, paraffin, mineral oils, silicone oils, vegetable oils, waxes, and the like.

The sweat permeable membrane may constitute a separate patch layer, but need not. For example, in one variation, the sweat-permeable membrane comprises an oil and/or petrolatum coating applied to the skin surface. In this way, only that glucose that comes to the skin surface via the eccrine sweat gland will be detected. Similarly, a liquid polymer coating, or a liquid bandage may be used as a sweat-permeable membrane. Typically, these materials are liquid membranes with low surface tension, which leave openings over the sweat gland pores when they cure (e.g., silicon polymers such as SILGARD®). The liquid polymer coating has significant advantages in that it is impermeable to water everywhere except the sweat gland pores, but a solid polymer layer with micropores may also be used, for example the Whatman NUCLEOPORE® polycarbonate track-etch membrane filters. Other suitable membranes include the ANOPORE® inorganic membranes consisting of a high-purity alumina matrix with a precise non-deformable honeycomb pore structure.

In some variations, it may be desirable to combine an adhesive polymer with the liquid polymers described above. In these variations, the liquid polymer would begin to cure (or set up as a solid) when exposed to oxygen (e.g., when the release liner is removed). The layer would cover the epidermis, but would leave holes only over the sweat gland orifices. In this way, only glucose brought to the skin surface via the sweat glands would be passed through to the collection layer. As noted above, in addition to allowing glucose in sweat to transport to the skin surface, the sweat-permeable membrane may also be useful in blocking diffusion and in blocking the generation of epidermal debris resulting from desquamation. Accordingly, only the glucose from the sweat, which can be correlated with blood glucose, will be measured.

The patch may also comprise an adhesive or an adhesive layer, for example, to help adhere the patch to the skin surface. The adhesive material may comprise an annular overlay layer or it may comprise a layer of adhesive contemporaneous and coextensive with at least one other patch layer. Any suitable adhesive may be used. For example, common pressure sensitive adhesives known in the transdermal patch arts, such as silicone, polyacrylates, and the like, may be used. We note here that in some circumstances, it may be desirable to provide an adhesive layer, or an adhesive and sweat-permeable barrier combination layer, that is relatively dry. This is because it is thought that excessive wetting of the stratum corneum may inhibit sweat gland function (see, e.g., Nadel, E. R. and Stolwijk, J. A. J., “Effect of skin wettedness on sweat gland response,” J. Appl. Physiol., 35, 689-694, 1973). In addition, the excessive wetting of the skin may help aid the liberation of glucose on the skin, resulting from desquamation. Accordingly, it may be desirable to limit the aqueous or otherwise wet nature of the interface between the skin and the patch.

While variations of patches containing adhesives have just been described, it is important to note that in some variations the patch does not comprise an adhesive. In these variations, the patch may be otherwise suitably adhered, held, or placed on the skin surface of a user. For example, the patch may be held on the skin surface by the user, or it may be held on the skin using an elastic material, medical tape, or the like.

The patch may also comprise a component to induce sweat by physical, chemical, or mechanical methods. For example, in one variation, the patch comprises pilocarpine with or without a penetration or permeation enhancer to induce sweat chemically or pharmacologically. The use of a penetration enhancer may help increase the rate at which the pilocarpine enters the body and thereby, increase the onset of the enhanced sweat response. Examples of suitable permeation enhancers include, but are not limited to ethanol and other higher alcohols, N-decylmethylsulfoxide (nDMS), polyethylene glycol monolaurate, propylene glycol monolaurate, dilaurate and related esters, glycerol mono-oleate and related mono, di and trifunctional glycerides, diethyl toluamide, alkyl or aryl carboxylic acid esters of polyethyleneglycol monoalkyl ether, and polyethyleneglycol alkyl carboxymethyl ethers. Pilocarpine may also be driven into the skin using iontophoresis. The present inventors have shown that the infusion of pilocarpine into the skin using iontophoresis increases the amount of sweat by about 20 fold per unit area. Similarly, other chemicals may be introduced into the skin to increase the sweat response.

The patch may also comprise a component that increases the sweat response by initiating a local temperature increase. For example, a heater (e.g., an electrical resistance heater) may be used to increase the skin surface temperature and thus increase sweating. Thermal induction of a sweat response may also be achieved by the application of energy (e.g., in the visible or near infrared regions). For example, a lamp may be used to generate heat and induce sweating. Experiments were run to measure the sweat rate (in μL/cm²×min) as a function of lamp power (W) versus time (sec). As shown by FIG. 4, there appears to be a minimum threshold required to induce a sweat response. In this instance, that threshold was in the range of about 2 to about 2.5 Watts (power to the lamp), when a MAGLITE®, Model LR00001, 6 Volt halogen lamp was used.

Direct electrical stimulation (i.e., Faradic stimulation) may also be used to induce a sweat response. Similarly, a chemical compound, or combination of compounds may be used to initiate a local temperature increase and therefore induce or increase the sweat response. For example, two chemical compounds may be used, separated by a thin membrane. The membrane may be removed by a pull-tab when the patch is adhered to the skin, thereby bringing the compounds into contact with each other, and causing an exothermic reaction. In this way, a source of heat is provided.

Physical mechanisms of inducing or increasing sweat may also be used. For example, in one variation, the measurement device, which will be described in more detail below with respect to the systems, is brought into contact with the patch and force is applied to the patch in a manner sufficient to cause an increase in the transport of sweat to the skin. The applied pressure over the collection patch results in fluid from the sweat gland lumen being expressed and delivered to the skin surface. In addition, the measurement device could include a suction or vacuum mechanism, which in combination with the applied pressure would result in a larger amount of sweat being delivered to the collection layer of the patch. Vibration may also be used to induce sweat.

Sweat may also be induced by the use of an occlusive layer within the patch, which inhibits evaporative loss from the skin surface and thereby permits a more efficient sweat accumulation into the patch collection layer. This occlusive layer may comprise an element within the patch, or may be a removable overlay which is separated from the patch prior to use of the measurement device. This occlusive layer may be, e.g., a thin polyvinyl film or some other suitable water vapor-impermeable material.

It should be understood that the patches may be of any suitable configuration or geometry. For example, they may have a rectangular geometry, a circular geometry, etc. The patch may also have a fun geometry, or include fun designs thereon (e.g., cartoons, shapes, dinosaurs, etc.), to entertain children. Similarly, the patch may be of any suitable size. For example, patches intended for the wrist will typically be larger than those intended for the fingertip. Typically, circular patches intended for use on the fingertip will have diameters in the range of about 1.0 cm to about 2.5 cm, or areas ranging from about 0.785 cm² to about 4.91 cm². For placement of the patch on other skin surfaces, the patch may have areas ranging from about 2 cm² to about 10 cm².

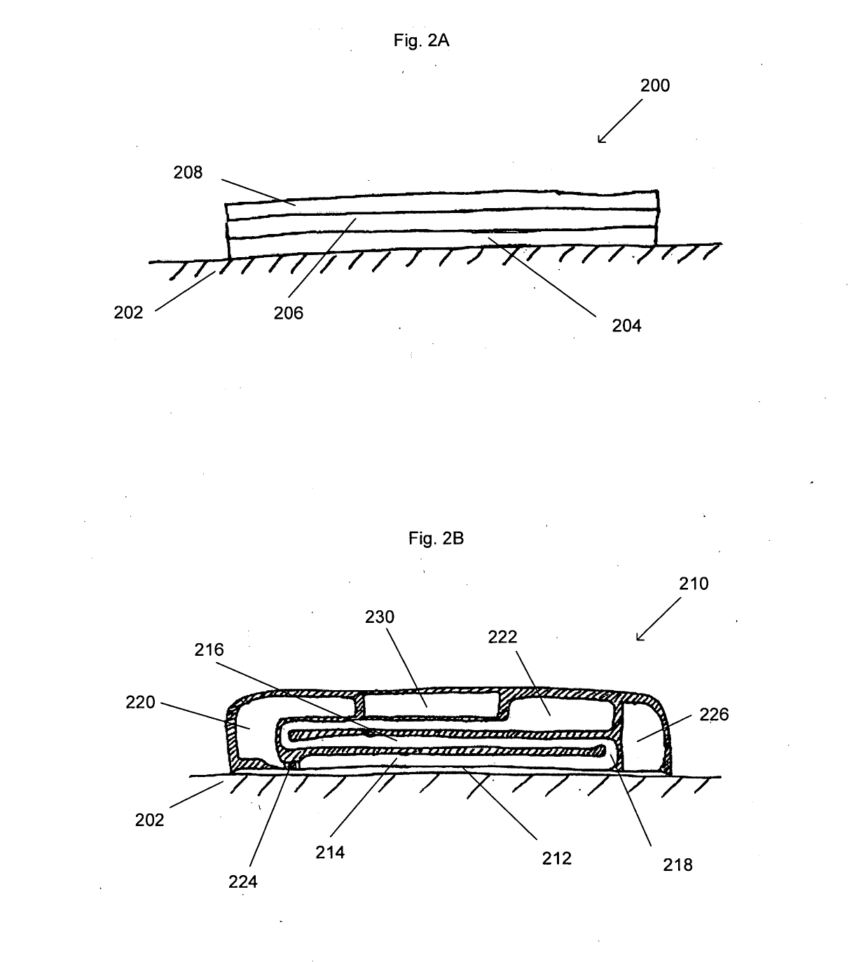

Making reference now to FIG. 2A, there is shown a cross-sectional view of patch (200) on skin (202). The patch (200) comprises an adhesive material in the form of a layer (204), a microfluidic collection layer (206), and a detector in the form of a detector layer (208). In some variations, the detector layer and the collection layer are in fluid communication with each other as shown in cross-sectional form in FIG. 2B. There, patch (210) comprises adhesive layer (212), collection layer (214), and a detector in the form of a detector layer (216). The collection layer (214) and detector layer (216) are in fluid communication with each other (218). As described in more detail below, the patch may also include a buffer and a buffer reservoir (220), a waste reservoir (222), and various microfluidic control features, such as valves (224), pumps, and the like. The patch may also include a device for measuring relative humidity (226).

While not shown in the figures, the patch may also include at least one release liner. For example, a release liner on the bottom adhesive surface would protect the adhesive layer from losing its adhesive properties during storage and prior to use. Similarly, a release liner may be placed on top of the patch to protect any optical or electrical components contained therein. In some variations, no release liner is used and the patch is topped with a backing layer. In some variations, the backing layer is made from a woven or non-woven flexible sheet, such as those known in the art of transdermal patches. In other variations, the backing layer is made from a flexible plastic or rubber.

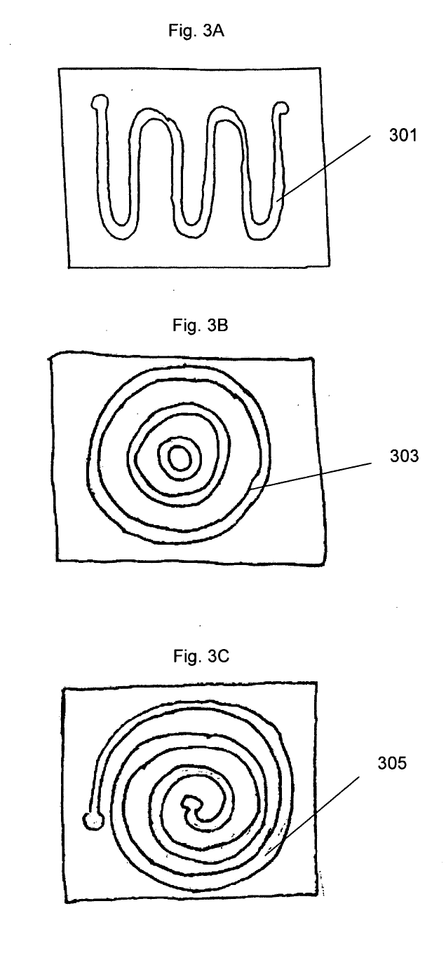

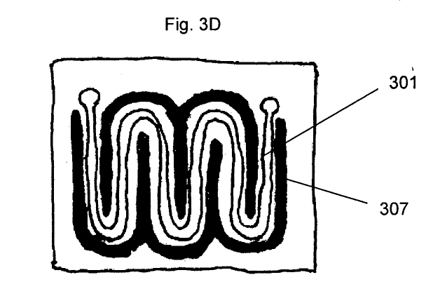

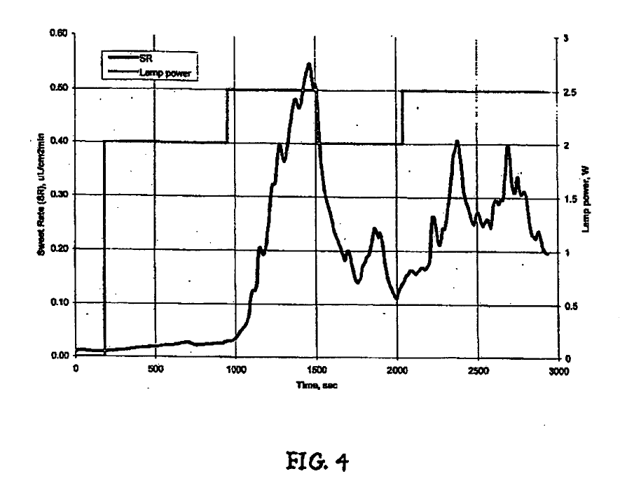

The microfluidic collection layer (214) may have a number of different configurations. In general, the microfluidic collection layer comprises one or more microfluidic channels. For example, the microfluidic collection layer may include a serpentine microfluidic channel (301), as shown in FIG. 3A, or it may comprise concentric microfluidic channels (303), as shown in FIG. 3B. In some variations, the microfluidic layer comprises a spiral microfluidic channel (305), as shown in FIG. 3C. Sweat may be collected within the microfluidic channel or channels. Serpentine and concentric microfluidic channels may maximize the surface area of the collection channel in contact with the subject's skin while also allowing movement of sweat and/or buffer through the channel. In some variations, sweat is collected into a substantially dry microfluidic channel. In other variations, sweat is collected into buffer that is present within the channel. The collection of sweat into the patch is described in greater detail below.

Sweat collected in the microfluidic channels is then typically moved from the collection layer into a detector layer. Additional microfluidic compartments (e.g., mixing compartments, treatment compartments, etc.) may also be included. The microfluidic channel may comprise a single channel, or multiple channels, and these channels may be connected. Similarly, the microfluidic channel or channels may be of any desirable and practical size (e.g., diameter or cross-sectional area) and length. The microfluidic channels may also be open to the skin, or they may communicate with the skin through a sweat-permeable membrane.

In some variations, the microfluidic collection layer is combined with a sweat-inducing layer, or one or more mechanisms for inducing sweat. For example, the microfluidic collection layer may include a mechanism for inducing sweat that acts mechanically (e.g., by using an occlusive backing layer, a vacuum, etc.), chemically (e.g., by administering sweat inducing agents such as pilocarpine with or without a penetration enhancer or iontophoresis), or thermally (e.g., by applying a heater, or initiating an exothermic chemical reaction, etc.). FIG. 3D shows the microfluidic layer of FIG. 3A with the addition of a mechanism for inducing sweat (307) at least partially surrounding the channel (301). In some variations, the mechanism for inducing sweat may be included within the microfluidic channel within the microfluidic collection layer. For example a buffer within the microfluidic channel may include a pilocarpine solution.

In some variations, it may be necessary to provide a method to minimize the effect of variable sweat rates on the amount of glucose accumulation in the collection layer. There are several ways in which the effect of variable sweat rates may be normalized by the method of collection or the use of various analytes. Measuring the relative humidity of the skin under the patch may allow determination of the sweat rate and therefore the amount of sweat collected.

One method of minimizing the effect of a variable sweat rate is to normalize the flux of the measured glucose. For example, when glucose is transported to the skin surface by sweat, the total amount of glucose deposited on the unit of skin surface per minute can be calculated as follows:
GF=SR×SG

where GF is glucose flux (ng/cm²×min), SR is the sweat rate (μL/cm²×min), and SG is the glucose concentration in sweat (ng/μL).

Often the sweat rate fluctuates over time as the result of physical or emotional stimulation, and this fluctuation can result in a variation in the amount of glucose collected from the skin surface, and hence the accuracy of the glucose concentration measurement. This variation can be significantly reduced if sweat rate is measured as a function of time and used to normalized the glucose flux, as follows:
GF/SR=(SR×SG)/SR=SG

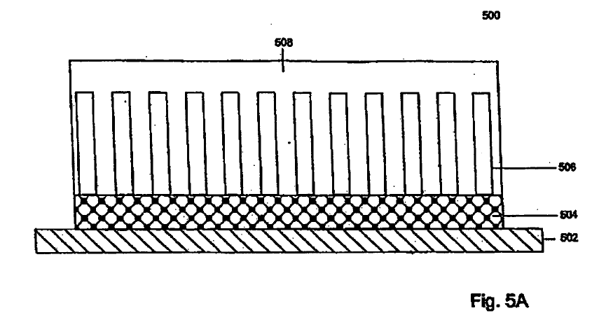

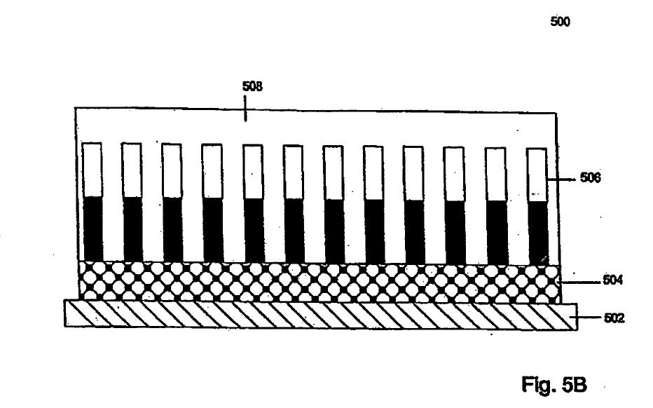

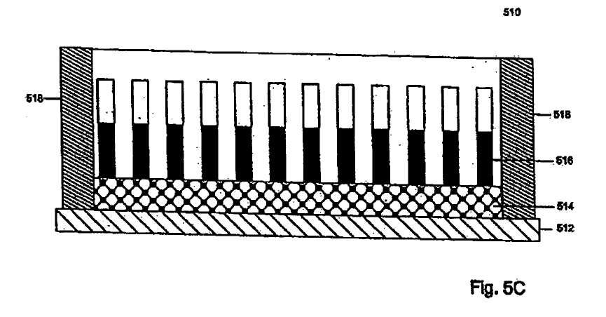

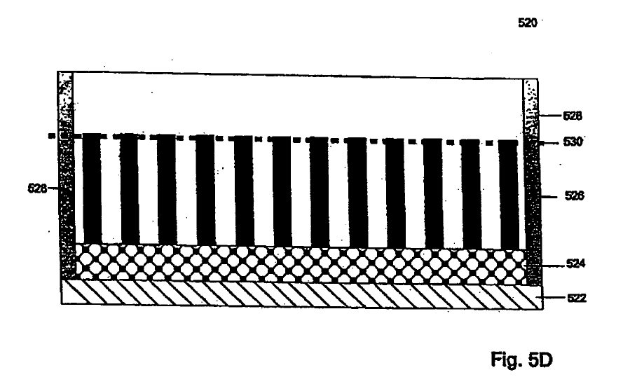

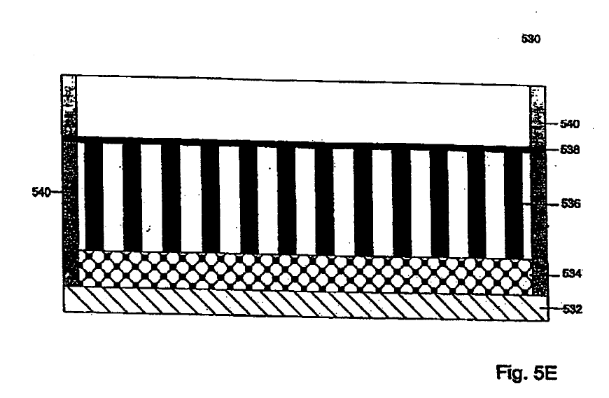

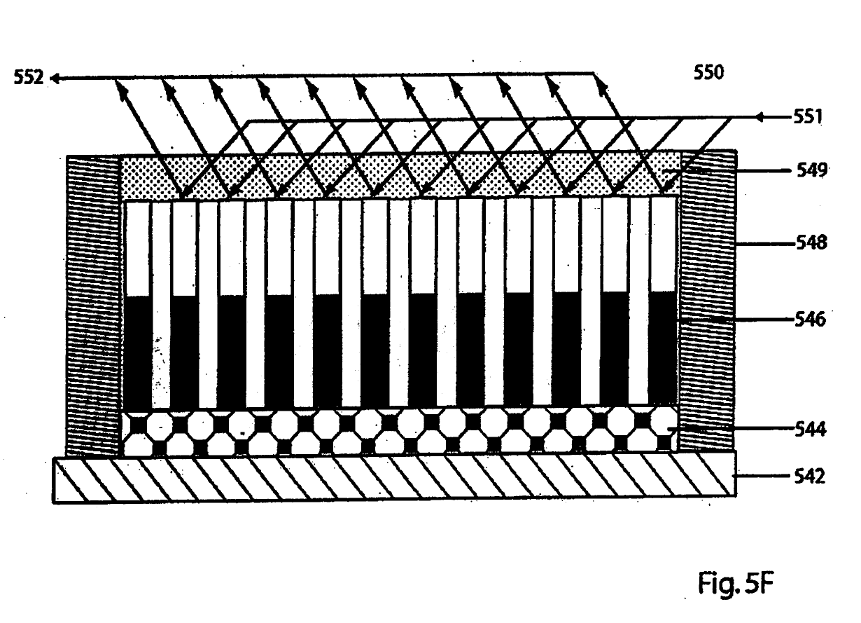

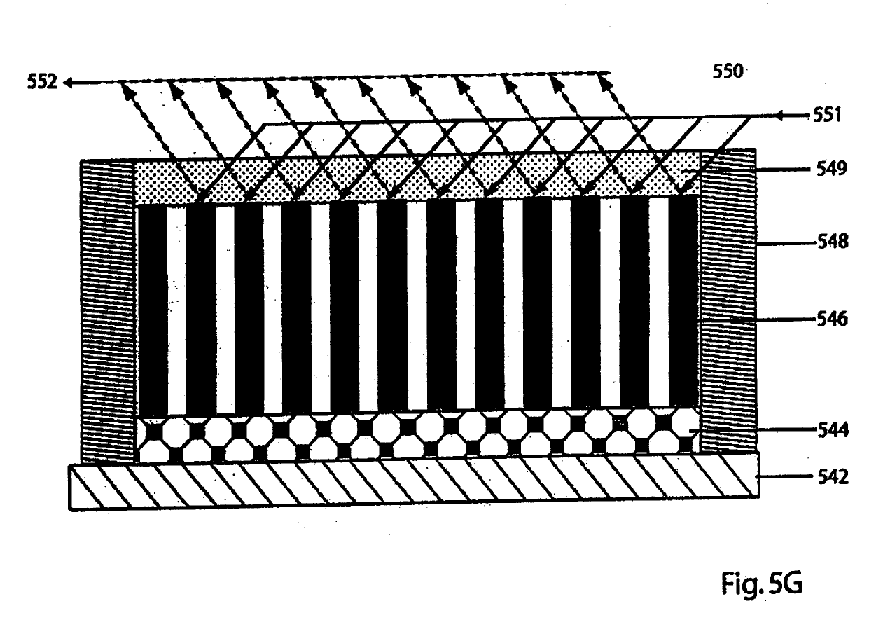

Another method, for example, may comprise configuring the microfluidic collection layer to collect a constant volume of fluid so that a variable sweat rate affects only the time to fill the collection volume, but not the amount of fluid collected. For example, the collection layer may comprise a reservoir having a fixed volume. FIG. 5A shows a patch (500) on skin surface (502). In this variation, the adhesive layer and the sweat-permeable membrane are combined in a single layer (504). Within the collection layer (508) is a fixed volume reservoir (506). The fixed volume reservoir (506) is shown in FIG. 5A as completely empty. As sweat begins to transport to the skin surface, and through the sweat-permeable membrane, the fixed volume reservoir begins to fill, as depicted in FIG. 5B.

A number of different techniques may be used to determine when the fixed volume reservoir, and hence the collection layer is filled. For example, electrical capacitance, electrical conductance, or optical measurements may be used as shown in FIGS. 5C, 5D, and SE respectively. For example, shown in FIG. 5C is patch (510) on skin surface (512). In this FIG., sweat has already passed through the adhesive and sweat-permeable membrane layer (514) to fill the fixed volume reservoir (516). Conductors (518) for forming a dielectric filled capacitor are placed on either side of the patch (510). In this way, the volume within the fixed volume reservoir (516) may be determined by a change in capacitance of the dielectric filled capacitor. Illustrative conductors suitable for use with the patches described herein include those made from silver, platinum, and the like.

Similarly, electrical conductance may be used to determine when the reservoir is filled. Shown in FIG. 5D is patch (520) on skin surface (522). Sweat has already passed through the adhesive and sweat-permeable membrane layer (524) to fill the fixed volume reservoir (526). A conducting circuit (530) is established with reservoir (526), here shown at the top of the reservoir. The circuit may be open or closed. In this way, the volume within the fixed volume reservoir (526) may be determined by a change in conductance (e.g., at the top of the reservoir). Supports (528) may be provided on either side of patch (520) to help provide structurally integrity thereto. These supports may be plastic substrates with suitably configured printed circuit elements that could provide a circuit path through the fixed volume reservoir. Changes in resistance or conductance at the top of the reservoir could indicate whether the fluid volume in the reservoir (or within the microfluidic channel) had reached a maximum. The modest power required to drive a current through the circuit described here could be provided by an inductive coupling mechanism enclosed within the measurement device, a plastic battery, and the like.

Optical transmission may also be used to determine when the reservoir is filled. Shown in FIG. 5E is patch (530) on skin surface (532). Sweat has already passed through the adhesive and sweat-permeable membrane layer (534) to fill the fixed volume reservoir (536). An optical transmission path (538) is established with reservoir (536), here shown at the top of the reservoir. In this way, the volume within the fixed volume reservoir (536) may be determined by a change in optical transmission (e.g., at the top of the reservoir). An optical fiber path could be provided at the top of the mechanical supports (540) on either side of patch (530) connecting an optical source on one side of the patch with an optical detector on the other. Changes in the measured transmission could indicate whether the fluid volume in the reservoir had reached a maximum. Power for the optical source and detector may be included in the measurement device.

Optical reflection may also be used to determine when the reservoir is filled. For example, as shown in FIG. 5F is patch (550) on skin surface (542). Sweat has already passed through the adhesive and sweat-permeable membrane layer (544) and partially filled fixed volume reservoir (546). A transparent plate (549) is located on the top of the reservoir. This plate has an optical index of refraction close to that of sweat (about 1.33). Incident light (551) illuminates the interface between reservoirs (546) and plate (549). Here, the reflected light (552) has a high intensity because the optical index difference between the plate (549) and air (which has an optical index of refraction of about 1.0) is high. Shown in FIG. 5G is the same patch (550) where the reservoir (546) is completely filled with sweat. Here, the reflected light (552) has a low intensity because the optical index difference between the plate (549) and sweat is low (both have an optical index of refraction of about 1.33). Thus, the drop in reflected light intensity may be used as an indicator that the reservoir is full. An optical source and detector may be included in the measurement device and the patch can be interrogated via an optical interface.

The determination of glucose level in the patch may be normalized for variable sweat rates by the use of a non-glucose analyte specific to sweat that is constant in concentration (e.g., lactate, urea, sodium chloride, other electrolytes, etc.). In this way, the glucose concentration may be normalized to that value. For example, a separate chemical detector may be incorporated into the patch to independently determine the amount of the sweat analyte. The amount of this sweat analyte accumulated in the collection layer depends only on the volume of sweat in the layer. Once this is determined, the amount of glucose measured in sweat may be normalized to the total volume of sweat collected, thereby avoiding errors associated with measuring an increased accumulation of glucose in the collection layer of the patch (i.e., due to increased sweating rather than increased physiological glucose concentrations). Alternatively, there may be physiological markers in sweat that increase with increased sweat rate. Determination of the concentration of these markers may also serve as a method for normalization of the glucose accumulated in the collection layer.

In some variations, the collection layer may be configured as a perfusion layer, wherein a buffer (e.g., phosphate buffered saline, or the like) is used to assist in the collection of sweat. For example, the collection layer may include a channel (e.g., microfluidic channel, tubing, etc.) or passage through which the buffer may be perfused.

Returning now to FIG. 2B, one variation of a patch includes a buffer reservoir (220) which may supply buffer to the microfluidic channel. The buffer reservoir may be part of the microfluidic layer, or it may be separate, but fluidly connected to the microfluidic layer. A pump may be connected to the buffer reservoir to move buffer from the reservoir through the patch (e.g., through the microfluidic collection layer and into and through a detector layer. Any appropriate pump may be used, including an active pump or a passive pump. An active pump actively applies pressure to move material (e.g., sweat, buffer, air, etc.) through the device. In general, the pump may be any pump compatible with the microfluidic channel. Examples of microfluidic pumps may include positional displacement pumps such as gear or peristaltic pumps, piezoelectric pumps, and membrane pumps.

Passive pumping methods may also be used (e.g., passive pumps). For example, material may be moved through the device by thermal pumps, osmotic pumps, or a preloaded pressure bolus. In one variation, buffer is moved through the device by allowing a pressurized bolus of buffer to enter the microfluidic channel and push sweat containing glucose from the collection layer into, and ultimately, through the detector layer. For example, buffer may be preloaded into the device under pressure. After sweat has collected in the microfluidic channel to an appropriate level (or for an appropriate period of time), the pressurized buffer is released from the buffer reservoir into the microfluidic channel so that the buffer moves through the microfluidic channel(s) in the collection layer, and propels the sweat into the detector layer. Buffer may be released from the pressurized buffer reservoir by any appropriate method, such as by activating a valve, or rupturing a membrane, etc.

FIG. 2B also illustrates a valve (224) separating the buffer reservoir (220) from the microfluidic channel in the collection layer (214). The flow of sweat, buffer, or other fluids (including gasses) through the device may be controlled by components such as valves, pumps, and switches, which may be controllable by a controller. Thus these components may include electronic or manual controls for regulating their operation. A controller may be part of the patch (230) or it may be separate from the patch (e.g., part of a measurement device, as described in more detail below).

The device shown in FIG. 2B also includes a waste reservoir for storing waste that has passed through the measurement device, such as sweat, buffer, etc. The waste reservoir may also include a pump (e.g., to draw material into the waste reservoir). Additional pumps may be used if desirable, to help control the movement of material through the device. Similarly, additional valves or switches may also be used if desirable. For example, a fluid connection between the collection layer and the detector layer may include a valve so that fluid (including sweat or sweat in buffer) does not enter the detector layer until the appropriate time.

As described above, the patch may comprise a detector. The detector may be in its own layer, adjacent to the collection layer, or, depending on the nature of the detector, it may be combined in the collection layer itself. In the absence of thermal, emotional, physical, or pharmacological stimulation, typical values of sweat output on the volar forearm and fingertip are relatively small. Sweat output varies from one individual to the next and from one anatomical site on the body to another. The maximum sweat rate per gland has been reported to range from about 2 nL/min to about 20 nL/min. See Sato, K. and Dobson, R. L. “Regional and individual variations in the function of the human eccrine sweat gland,” J. Invest. Dermat., 54, 443, 1970. Assuming insensible perspiration rates per gland of 1 nL/min and using measured sweat gland densities at different parts of the body, a total sweat output can be estimated. Typical sweat gland densities on the forearm are approximately 100 glands per square centimeter, which give 0.1 μL sweat per square centimeter per minute. Typical sweat gland densities on the volar fingertip are approximately 500 glands per square centimeter, which give 0.5 μL sweat per square centimeter per minute. In the absence of stimulation, the number of active sweat glands per unit area is often reduced by one-half the total available. Boysen et al., described above, found that the glucose concentration in sweat was approximately one one-hundredth normal blood glucose values (e.g., 1 mg/dl). Hence the flux of glucose to the surface of the volar fingertip may be estimated to be in the range of from about 2.5 nanograms to about 5 nanograms per square centimeter per minute. The flux to the surface of the volar forearm or wrist is likely to be even lower. Accordingly, the detector described here must be capable of detecting nanogram quantities of glucose and the measurement device described herein must be capable of performing ultra-sensitive glucose measurements.

Indeed, we have demonstrated that the flux of glucose brought to the skin via sweat was on the order of 1-20 nanograms per square centimeter per minute in the absence of thermal, pharmacological or other forms of stimulation. These measurements were made using the Wescor MACRODUCT® (459 South Main Street Logan, Utah 84321) system and in specially adapted sweat collection chambers. Sweat collected in the Wescor MACRODUCT® and in the sweat collection chambers was then analyzed using a Dionex (Sunnyvale, Calif.) High Performance Anion Exchange with a Pulsed-Amperometric Detector (HPAE-PAD). The sensitivity and specificity of the HPAE-PAD system was tested using analytical samples. We detected glucose in amounts as low as 1 nanogram using HPAE-PAD.

Several types of suitably sensitive detectors may be used. For example, the detectors may be electrochemical-based, or may be fluorescent-based. Suitable electrochemical sensors may be those comprising an immobilized glucose-oxidase or other enzyme(s) in or on a polymer or other support, and those comprising glucose-oxidase or other enzyme(s) in a microfluidic configuration. Similarly, the detector may be fluorescent-based, for example, based on enhanced or suppressed fluorescence of a glucose-sensitive fluorescent molecule. The detector may be immobilized within a layer, or may be in solution.

As noted above, any suitable electrochemical detector may be used. For example, the electrochemical detector may be polymer based, based on microfluidics, and the like. When the electrochemical detector is polymer based, the polymer is typically permeable to glucose, and a glucose-reactive enzyme is immobilized on or within the polymer. In these variations, the detector typically comprises at least two electrodes, which are typically activated by the measurement device when it is brought into electrical contact with the patch. In one variation, the enzyme glucose oxidase is used, which produces hydrogen peroxide that reacts at the at least one electrode to produce a measurable electrical current proportional to the glucose concentration. That is, using an enzymatic process known in the art, the glucose oxidase catalyzes the reaction of glucose and oxygen to produce gluconic acid and hydrogen peroxide. The hydrogen peroxide is then electrochemically reduced at the at least one electrode, producing two electrons for detection. Electrical contact between the measurement device and the patch may also serve to provide power to the patch (although, the patch may comprise a battery therein as well if needed). The measurement device, which will be described in more detail below, interrogates the patch (i.e., the detector) and provides a glucose measurement reading.

When microfluidics based electrochemical detectors are used on the patch, the patch typically comprises a fluid reservoir, a flow channel, a gating valve, and sensor electrodes. In this variation, the electrochemical enzyme is typically in solution. The interface layer comprises at least one electrode, which is activated by the measurement device when placed into electrical contact with the patch. As with the case above, electrical contact between the measurement device and patch, may serve to power the patch. A microfluidic sensor may also comprise a reservoir with a reference analyte to provide in situ calibration of the detector. As with the cases above, electrical contact between the measurement device and patch, may serve to provide power to the patch, or the patch may comprise a battery therein.

Sensitivity to these electrochemical detectors may be increased by increasing the temperature during the detection cycles, by increasing the length of the detection cycle, by increasing the area of the detector, by appropriately selecting the operating potential, and by the use of selective membranes to screen interfering substances such as ascorbic acid, uric acid, acetaminophen, etc. In addition, differential methods may be used where the glucose sample is measured in the presence and absence of a glucose-specific enzyme and the glucose concentration is determined from the difference between these two signals.

For example, sensitivity may be increased by heating the sensor solution from 25° C. to 40° C., and such temperature increase is unlikely to affect the enzyme activity of the glucose detector. See, e.g., Kriz, D, Berggre, C., Johansson, A. and Ansell, R. J., “SIRE-technology. Part I. Amperometric biosensor based on flow injection of the recognition element and differential measurements,” Instrumentation Science & Technology, 26, 45-57 (1998). Similarly, sensitivity may be increased by increasing the area of the detector, since the detector current increases linearly with the area of the detector electrode. Extending the length of time over which the measurement may be made may also be used to increase the measured charge and hence, the overall sensitivity of the detector. Lastly, covering the electrode with size- and, or, charge-selective membranes can allow passage of hydrogen peroxide, for example, while excluding ascorbate, urate and other material, which can react directly with the sensor to produce a spurious signal. Suitable size-selective membranes, for example, include those made of polyurethane, polyethylene and other materials as well as charge-selective membranes made of polyethylsulfide, NAFION®, cellulose acetate, and other materials that can be used as interference-screening membranes for electrochemical detectors.

As noted above, the detector may also be a fluorescent detector. In this variation, the detector layer, or the layer immediately adjacent to the measurement device may be made of a material that is optically transparent at the relevant excitation and emission wavelengths for the particular fluorescent-based detector used by the patch. In one variation, the measurement device need not be brought into direct physical contact, because interrogation of the patch is achieved by optically coupling the device and patch. The internal electronics of the measurement device may also be configured to record a maximum signal as it is passed over the patch, thereby reducing the need for proper static registration between the measurement device and the patch itself. The patch may also include a glucose-insensitive reference fluorescent molecule to provide a ratiometric, rather than an absolute intensity measurement. The addition of a reference molecule may also protect against a spurious signal originating at the emission wavelength of the fluorescent-based detector.

When a fluorescent detector is used, it typically comprises a glucose-sensitive fluorescent molecule immobilized in a polymer or suitable solvent, and as described above, may be in a separate layer, or dispersed throughout the collection layer. Because the measurement device will be measuring the glucose at a specific wavelength, it is desirable that the materials used in the patch do not have fluorescence at, or substantially near, the wavelength of the fluorescent emission of the glucose transducer molecule. Similarly, it is often desirable that the sweat-permeable membrane in these variations be opaque so as to prevent autofluorescence from the skin.

Suitable fluorescent detectors for example may be those described in U.S. Pat. No. 6,750,311 to Van Antwerp et al, which section on fluorescent detectors is hereby incorporated by reference in its entirety. As described there, fluorescent detectors may be based on the attenuation in the fluorescence intensity of labeled lectins or boronate (germinate or arsenate) aromatic compounds. Suitable lectins include concanavalin A (Jack Bean), Vicia faba (Fava Bean), Vicia sativa, and the like. Such lectins bind glucose with equilibrium constants of approximately 100. See, Falasca, et al., Biochim. Biophys. Acta., 577:71 (1979). The lectin may be labeled with a fluorescent moiety such as fluorescein isothiocyanate or rhodamine using commercially available kits. The fluorescence of the labeled lectin decreases with increasing glucose concentration.

Boronate based sugar binding compounds may also be used as the basis for the fluorescent detector. Glucose reversibly binds to the boronate group in these compounds. Boronate complexes have been described which transduce a glucose signal through a variety of means. See, Nakashima, et al., Chem. Lett. 1267 (1994); James, et al., J. Chem. Soc. Chem. Commun, 477 (1994); and James, et al., Nature, 374:345 (1995). These include geometrical changes in porphyrin or indole type molecules, changes in optical rotation power in porphyrins, and photoinduced electron transfer in anthracene type moieties. Similarly, the fluorescence of 1-anthrylboronic acid has been shown to be quenched by the addition of glucose. See, Yoon, et al., J. Am. Chem. Soc., 114:5874 (1992).

The dye used in the above fluorescent-based detector may be, for example an anthracene, fluorescein, xanthene (e.g., sulforhodamine, rhodamine), cyanine, coumarin (e.g., coumarin 153), oxazine (e.g., Nile blue), a metal complex or other polyaromatic hydrocarbon which produces a fluorescent signal. Unlike previously described applications of these sensors, where the sensors are specially-designed for equilibrium-binding with a target analyte and for reversibility, the binding constant of the fluorescent-based detectors described here may be increased so as to further lower the limit of detection.

Measurement Device

As noted above, the glucose monitoring systems described here generally comprise a patch configured to collect a nanogram quantity of glucose in sweat, where the patch comprises a microfluidic collection layer and a detector, and a measurement device configured to measure the nanogram quantity of glucose collected. The patches were described in detail above.

The measurement device interrogates the patch to measure glucose. The device measures the total quantity of glucose present in a fixed volume, and then converts the glucose measurement into a concentration. The measurement device may comprise a display, to display data. The device may also include warning indicators (e.g., a word prompt, flashing lights, sounds, etc.) to indicate that a user's glucose levels are dangerously high or dangerously low. In addition, as described briefly above, the measurement device may also be configured to verify that a skin-cleaning procedure has been performed. For example, when wipes with a marker have been used, (which will be described in more detail below) the marker remains on the skin surface. If the measurement device detects the marker, then the measurement proceeds. If the measurement device does not detect the marker, the measurement does not proceed. The measurement device may also comprise an iontopheric source, for example, to be used to help drive pilocarpine, or other molecules of interest into the skin.

In general the configuration of the measurement device is dependent on the configuration of the detector in the patch. For example, when the measurement device is to be used with an electrochemical detector, the measurement device provides an electrical contact with the interface layer, and is either powered by the electrical contact, or is powered by an independent power source (e.g., a battery within the patch itself, etc.). The measurement device also typically comprises a computer processor to analyze data. Conversely, when the measurement device is configured for fluorescence detection, the measurement device is configured to provide optical contact or interaction with the interface layer. In this variation, the measurement device also typically comprises a light source to stimulate fluorescence. In some variations, the measurement device comprises both the necessary electrical contacts and the necessary optics so that a single measurement device may be used with a patch having various configurations of patch layers (e.g., one layer comprising a fluorescent-based molecule, and another layer comprising an electrochemical detector).

The measurement device may further comprise computer executable code containing a calibration algorithm, which relates measured values of detected glucose to blood glucose values. For example, the algorithm may be a multi-point algorithm, which is typically valid for about 30 days or longer. For example, the algorithm may necessitate the performance of multiple capillary blood glucose measurements (e.g., blood sticks) with simultaneous patch measurements over about a 1 day to about a 3 day period. This could be accomplished using a separate dedicated blood glucose meter provided with the measurement device described herein, which comprises a wireless (or other suitable) link to the measurement device. In this way, an automated data transfer procedure is established, and user errors in data input are minimized.

Once a statistically significant number of paired data points have been acquired having a sufficient range of values (e.g., covering changes in blood glucose of about 200 mg/dl), a calibration curve will be generated, which relates the measured sweat glucose to blood glucose. Patients can perform periodic calibration checks with single blood glucose measurements, or total recalibrations as desirable or necessary.

The measurement device may also comprise memory, for saving readings and the like. In addition, the measurement device may include a link (wireless, cable, and the like) to a computer. In this way, stored data may be transferred from the measurement device to the computer, for later analysis, etc. The measurement device may further comprise various inputs, to control the various functions of the device and to power the device on and off when necessary.

As mentioned above, the system may also include a device for measuring the relative humidity of the skin under the patch, which may or may not be part of the measurement device (e.g., it may be part of the patch as shown above in FIG. 2B). The relative humidity may provide an estimate of the amount of sweat collected by the device, or the rate of sweat over time. Any appropriate relative humidity detector may be used. In some instances, it may be desirable to use full range (e.g., 0% to 100%) relative humidity sensors. Examples of appropriate relative humidity sensors include capacitive humidity sensors, resistive humidity sensors, and low-voltage humidity sensors. The relative humidity measured beneath the patch reflects the amount of moisture lost by the skin (e.g., sweat) and therefore the amount and rate of sweating.

As mentioned above, the measurement device may also include a controller for controlling the patch or its components (e.g., valves, pumps, switches, etc.). In some variations, the controller regulates the movement of fluid (e.g., sweat, buffer, and/or air) through the collection and detector layers. A controller may achieve this by coordinating the activity of pumps, valves, and switches. For example, the controller may open the connection (e.g., a switch or valve) between the buffer reservoir and the microfluidic channel and pump buffer from the buffer reservoir into the microfluidic channel. Buffer may be added to the microfluidic channel either before the collection of sweat (e.g., in “wet” collection procedures) or after sweat has been collected (in “dry” collection procedures). One or more switches may be used to switch between different regions of the patch. For example, a marker such as a bolus of gas, buffer, or marking solution may be applied to one end of the microfluidic collection channel by opening a channel between the source of marker material and the end of the microfluidic channel. Another switch may also control the movement of material from the microfluidic collection chamber to the detector layer. For example, when the collection layer comprises a microfluidic channel into which sweat is collecting, the distal end of the channel may be open to a reservoir or to the atmosphere, preventing a backpressure within the channel. After an appropriate amount of sweat has been collected, a valve or switch may switch the microfluidic channel so that it is instead in fluid communication to the detector layer, allowing sweat (including sweat in buffer) and other material in the microfluidic channel to pass into the detector layer. Sweat may be pumped (passively or actively) from the collection layer into the detector layer so that the level of glucose may be determined.

The measurement device may be worn by the user, but need not be. For example, since the patches described here are suitable for both single and repeated measurements, it may be desirable in some circumstances to have the measurement device be wearable. For example, in the case where the patch will be interrogated multiple times, as will be described in more detail below, the measurement device may be worn over the patch in a bracelet or watch-type configuration. In these variations, the measurement device should be of a size suitable to provide comfort to the wearer, while at the same time being capable of housing its necessary components. It should be understood that the size of the measurement device and how it is configured for comfortable wear is also dependent upon the patch location (e.g., fingertip, wrist, forearm, abdomen, thigh, etc.).

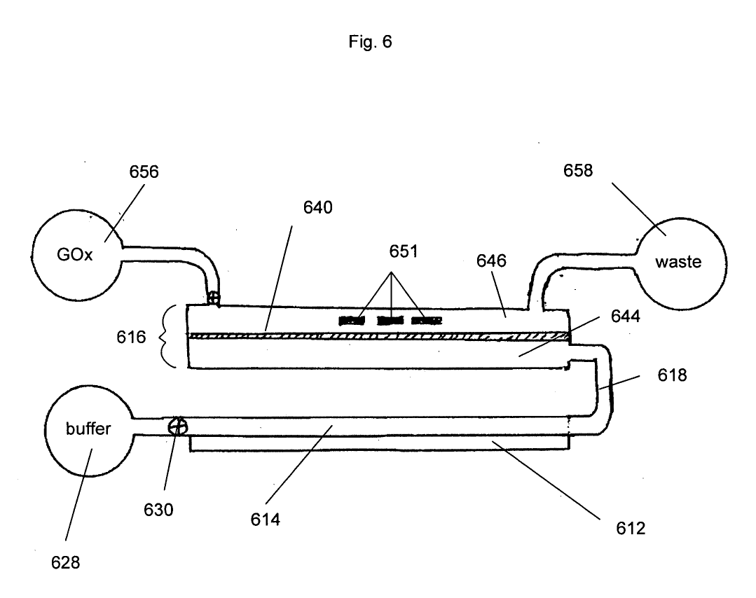

An exemplary depiction of a glucose monitoring system as described herein is shown in FIG. 6. FIG. 6 shows a patch configured as an in-line glucose detection device that uses glucose oxidase (“GOx”) in solution as part of an electrochemical detector. In this variation, the device uses a differential measurement technique to enhance the glucose signal while eliminating potential contaminants.

In the system illustrated in FIG. 6, a sweat sample from the skin is collected into the microfluidic collection layer of the patch (614). The collection layer comprises a microfluidic channel, which may be a serpentine channel, as described above. In this example, the device includes a sweat permeable membrane (612) between a user's skin and the collection layer (614). The distal end of the microfluidic chamber is in fluid connection with a source of buffer, such as a buffer reservoir (628). The buffer may be pressurized (e.g., by a pump) so that when the valve (630) between the buffer reservoir and the channel is opened, buffer flows into the channel.

As described above, a buffer (which may or may not be different from the buffer in the buffer reservoir) may be preloaded into the microfluidic channel so that sweat is collected into fluid within the microfluidic channel. In some variations, the sweat is collected into a relatively “dry” channel. Typically buffer entering the microfluidic channel from the buffer reservoir will drive material (e.g., sweat) from the microfluidic channel and into the detector region (616). In some variations, fluid in the microfluidic channel is pumped into the detector region (616) by air or by a material other than the buffer (including immiscible materials such as oils, etc.) that is added at the proximal end of the microfluidic channel. In some variations, this may serve to mark the end of the material collected into the microfluidic channel as it passes into the detector. As shown in FIG. 6, the collection layer is fluidly connected to the detector region by tubing (618). An additional valve may be used to separate the detector layer from the collection layer.

As mentioned above, different buffers may be used as part of the same system. For example, a collection buffer may be used to collect sweat, and a different buffer (e.g., a pushing buffer) may be used to move a sweat sample (and/or collection buffer) within the system. A different marker buffer may be used to “mark” the microfluidic solution. In some variations, the same buffer may be used for all of these. These buffers may have the same ionic strengths and pH, or they may have different ionic strengths and pH. In some variations, the same buffer is used for all of these different applications.

As mentioned above, the detector shown in FIG. 6, is a GOx based detector that applies a differential detection method to measure glucose. In this way, glucose can be accurately measured even in the presence of additional compounds such as ascorbic acid and acetaminophen that might otherwise inhibit or interfere with an accurate measurement. Here, the detector layer is divided up into two separate regions by a dialysis membrane (640) that allows glucose to pass therethrough, but prevents larger molecules (such as GOx) from passing. An appropriate differential measurement technique is described in U.S. Pat. Nos. 6,706,160 and 6,214,206, both of which are herein incorporated by reference in their entirety. Differential measurement methods typically remove the impact of interfering substances by recording from a sweat sample in the presence and absence of GOx, and producing a differential signal.

In FIG. 6 the detector layer comprises two regions (644, 646) separated by the dialysis membrane (640). The upper region (646) contains three electrodes (651): a working electrode, a counter electrode and a reference electrode. This upper region is also in fluid communication with a source of GOx (565), and a waste reservoir (658). In some variations, (particularly non-differential measurement variations) the GOx may be fixed or immobilized (e.g., to the sides of the detection region, or on or near the electrodes), rather than applied in solution.

The sweat collected into the microfluidic channel may be passed (in-line) into the lower region of the detector (644), as shown. Once the sweat sample enters the detection chamber from the collection region, a signal may be measured from the electrodes (e.g., a working electrode and a counter electrode pair). The typical sweat sample may contain other non-glucose substances (such as ascorbic acid and acetaminophen) that can generate a signal on an electrode, resulting in a background current. These compounds may also pass through the dialysis membrane (640) between the upper and lower regions of the detector layer, and would be present as background in an electrochemical signal. However, as mentioned previously and will be described in more detail below, because a differential measurement technique is used, the background signal of the potentially interfering compounds is of no consequence.

As mentioned previously, glucose is also free to diffuse across the dialysis membrane (640) between the upper and lower chambers. To measure the glucose concentration, GOx is then added (e.g., from the GOx reservoir (656) into the upper chamber where it can react with glucose and produce a signal proportional to the glucose concentration on the electrodes. The enzyme does not pass through the dialysis membrane (640), and converts glucose into peroxide resulting in a “peroxide current” local to the upper chamber electrodes. The difference in the signals before and after the addition of GOx may accurately reflect the concentration of glucose even in the presence of interfering compounds.

The signal present at the electrodes (651) may be monitored and used by the measurement device (not shown), as described above. Furthermore, the coordination of the taking of measurements, addition of GOx, etc. may be performed by a controller, including a controller that is part of the patch, or part of the measurement device.

In some variations, the detector layer comprises a GOx detector that is not a differential detector. Thus, the system shown in FIG. 6 may be simplified by removing the dialysis membrane (640) and reducing the upper and lower regions (644,646) into a single region. This may be particularly desirable if the levels of potentially interfering compounds is low.

Methods

As noted above, methods for measuring glucose on the skin surface are also provided here. Some methods generally comprise cleaning the skin surface with a glucose solvent, collecting sweat from the skin surface using a microfluidic collection device, and measuring the collected glucose.

Cleaning the skin surface (e.g., by wiping it clean) is typically performed to remove any “old” or residual glucose remaining on the skin. In variations using a wipe, the wipe is typically made of a material suitable for wiping the skin and comprises a solvent for removing glucose. For ease of description only, the term “wipe” will be used herein to include any type of fabric, woven, non-woven, cloth, pad, polymeric or fibrous mixture, and similar such supports capable of absorbing a solvent or having a solvent impregnated therein.

In some variations, the wipe contains a marker that is deposited on the skin. In these variations, the measurement device looks for the presence of the marker, and if the marker is detected, then the measurement proceeds. If the marker is not detected, the measurement does not proceed. In some variations, as will be described in more detail below, the measurement device provides an indication to the user that the skin has not been wiped. In this way, the possibility that a user obtains and relies upon a clinically dangerous measurement (e.g., based on an erroneous reading resulting from food residues or other glucose sources on the skin that are not correlated with the user's actual blood glucose) is minimized, and accurate measurements are facilitated. The marker may comprise a chemical having a short half-life, so that it will decay after a short period of time. In this way, a marker will only be valid for a single wipe, or a single use and erroneous detection of a marker on the skin surface will be minimized. In a like manner, the marker may also be bound to a volatile compound, and made to evaporate in a short period of time.

It should be noted however, that the wipe should not contain solvents, markers, or other chemicals that would interfere with the measurement of glucose. That is, a suitable glucose solvent would have the capacity to solubilize glucose without interfering with either the electrical or optical measurement of glucose. Polar solvents, and in particular, a mixture of distilled water and alcohol, have provided very good results in removing residual glucose from the skin surface. The ratio of distilled water to alcohol may be chosen such that there is sufficient water to dissolve the glucose, but not so much water as to make the removal of the excess water take an inconveniently long period of time relative to the measurement of glucose (e.g., more than 25 minutes). As noted above, it is desirable that the alcohol/water mixture, or other polar solvent, be selected such that it removes the residual glucose, but does not interfere with the glucose measurement.

In some variations, the skin is cleaned by rinsing or otherwise treating it with a glucose solvent to remove potentially contaminating residual glucose. After cleaning the skin, it may be dried (or allowed to dry), removing excess cleaning solution. A separate drying step is unnecessary in some variations.

As noted above, after the skin has been cleaned, sweat is collected from the skin surface, and this may or may not include placing a patch on the skin surface for sweat collection. When a patch is used, it may be placed on any suitable skin surface as described briefly above. For example, the patch may be placed on a finger, on the palm, on the wrist, the forearm, the thigh, etc. Placement of the patch on the fingertip may provide a convenient, discrete, and readily accessible site for testing, particularly non-continuous testing. In addition, fingertips have the greatest density of sweat glands. Placement of the patch on the wrist may provide a convenient, discrete, and readily accessible site for testing when repeated measurements are to be taken from a single patch.

These methods may also include a step of inducing sweat prior to collecting the sweat from the skin surface. The step of inducing sweat may comprise inducing sweat mechanically, chemically, physically, or thermally, as described in detail above. In some variations, measuring comprises measuring nanogram quantities of glucose.

Other methods for measuring glucose on the skin surface comprise cleaning the skin surface with a glucose solvent, as described just above, collecting sweat from the skin surface in a patch comprising a microfludic collection layer; and measuring glucose collected in the patch. Again, any of the patch variations described above may be used with the patch described here. In some variations, collecting sweat comprises collecting sweat in a microfludic collection layer containing a buffer. For example, the patch may be applied to a user's skin, and the microfluidic channel may be filled (or it may have been pre-filled) with a buffer. In some variations, the buffer includes a mechanism for inducing sweat (e.g., pilocarpine). Sweat is therefore collected into the buffer solution within the microfluidic collection channel. After an appropriate amount of sweat has been collected, the buffer within the collection channel is pumped into the detector layer. The appropriate amount of sweat may be determined based on any of the methods descried above. For example, the appropriate amount of sweat may be determined by the volume of sweat collected (e.g., when the sweat added to the buffer within the collection layer increases by a given amount), or based on the concentration of another component of the sweat detected while in the collection channel, or based on the rate of sweat determined by the relative humidity of the skin beneath the patch, or based on a predetermined lapse of time.

The sweat (in the buffer) may be moved into the detector layer from the collection layer. Sweat may be pumped by applying pressure at the proximal end of the microfluidic collection passage, when the collection layer is in fluid communication with the detector layer. Pressure may be applied by adding additional buffer to the proximal end of the collection layer, or by adding any appropriate material (e.g., air, etc.). Once in the detector layer, the concentration of glucose in the sweat may be determined by any appropriate method, as described above. The detection may occur while the material is entering into the detector layer (e.g., continuously), or it may be done at a discrete time periods after the sweat has entered. The measurement device may interrogate the detector as (or after) the sweat has entered the detector layer. Thus, the measurement device may sample the detector to determine the concentration of glucose. As described above, in some variations, the measurement device may apply a differential technique to determine a glucose signal, or it may average, sum, or otherwise analyze the output of the detector to determine a glucose concentration that reflects the concentration of blood glucose. The sweat (and/or buffer) in the detector layer may be pumped past the detector (e.g., electrodes) and into a waste reservoir.

The method may also include pumping a buffer into the microfluidic collection layer (e.g., after collecting the sweat). In these variations, the patch typically has a collection layer and a detector layer, which are in fluid communication with each other. Sweat may be collected into an initially relatively dry microfluidic collection layer. A sufficient amount of sweat may then be collected before moving the sweat into the detector layer. As mentioned previously, the amount of sweat collected may be measured by the device in any appropriate fashion. Any of the steps previously described may then be used to determine the concentration of glucose in the sweat.

Of course, it should be understood that any of the steps of the methods described herein may be repeated (e.g., collecting the sweat and measuring the glucose). Thus, the devices described herein may be configured for repeated measurements of glucose from sweat.

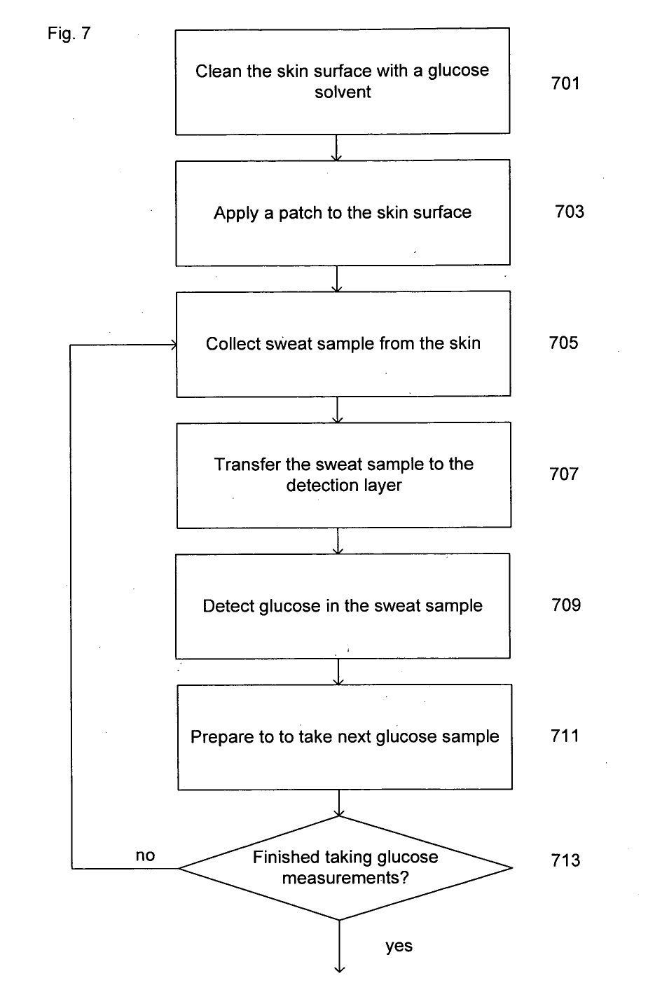

Still other methods for measuring glucose on a skin surface comprise cleaning the skin surface with a glucose solvent, as described above, collecting a first sweat sample from the skin surface in a patch comprising a microfludic collection layer and a detector layer, transferring the first sweat sample from the collection layer to the detector layer, measuring glucose in the first sweat sample, and repeating the collection, transferring, and measuring steps at least once. This method is shown in flow chart form in FIG. 7.

In FIG. 7, one example of a method for repeatedly measuring glucose from sweat is depicted. The subject's skin is first cleaned (701), as described above, with an appropriate glucose solvent, and then the patch is applied (703). Any appropriate skin region may be used, preferably a region to which the patch and/or measuring device (e.g., monitor) may be attached for the period of time over which repeated measurements are to be taken (e.g., minutes, hours, days). For example, the patch may be applied to the subject's wrist, abdomen, arm, etc.

A first sweat sample may then be collected from the skin surface (705), according to any of the methods described herein. During or before the collection of sweat, a mechanism for inducing sweat may be applied to induce a sweat response from the skin. For example, the mechanism for inducing sweat may be chemical (e.g., pilocarpine with or without penetration enhancers or iontophoresis), thermal (e.g., heater), or mechanical (e.g., occlusive layer).

Sweat may be collected through a sweat-permeable membrane (but need not be) into a microfluidic channel, such as a serpentine microfluidic channel. As described above, the step of collecting the first sweat sample may comprise collecting the first sweat sample in a microfludic collection layer containing a buffer or may comprise collecting the first sweat sample in a microfluidic collection layer devoid of a buffer. In one variation, the microfluidic collection layer includes a buffer (e.g., PBS at pH 7.4) into which sweat is collected. Sweat may be collected for an appropriate amount of time, or until an appropriate amount of sweat has entered the microfluidic channel. In one example, the appropriate amount of sweat is determined based on the displacement of fluid within the microfluidic channel. For example, as sweat enters the buffer within the channel, the volume of fluid (buffer plus sweat) within the channel will increase, and this increase may be detected by the device, using any of the methods previously described. For example, when the end (closest to the entrance of the detector layer) of the microfluidic channel is blocked, the addition of sweat to the buffer will extend the front of the buffer within the microfluidic chamber, which may be detected optically, electrically, etc. In some variations, an end of the microfluidic chamber is open to atmosphere via a valve or switch, so that backpressure does not develop. Examples of the appropriate amount of sweat collected may be less than about 20 μl, less than about 10 μl, less than about 5 μl, less than about 1 μl or less than about 0.5 μl.

After the first sweat sample has been collected, the sweat sample (in buffer) may then be transferred from the microfluidic collection layer into the detector layer (707). As described above, any appropriate method may be used to transfer the sweat and buffer into the detector layer. For example, the step of transferring the first sweat sample from the collection layer to the detector layer may comprise pumping a buffer into the microfluidic collection layer or may comprise applying pressure (e.g., gas pressure, liquid pressure, or mechanical pressure) within the microfludic collection layer. In some variations, pressure is used to transfer the sweat sample and pressure is applied with pressurized saline. Other variations for transferring the sweat sample may also be used. Pressure is typically applied within the microfluidic collection channel when the channel is in fluid connection with the detector layer. In one variation, additional buffer is pumped into the proximal end of the microfluidic collection layer from a buffer reservoir after opening a valve to the buffer reservoir from the microfluidic channel, while also opening a valve between the microfluidic channel and the detector layer.

Once the sample is in the detector layer, the concentration of glucose may be determined (709) according to any of the methods previously described (e.g., electrochemically, fluorescently, etc.). Thus, if an electrochemical method is used with GOx, the GOx may react with glucose in the sample to produce a current that is proportional to the concentration of glucose even at very low (e.g., nanogram) levels, as previously described.

After the glucose reading has been taken, the remaining sample may be driven (e.g., by pressure) into a waste reservoir, and the device may be in preparation for collecting the next sample (711). For example, the microfluidics channel may be purged with air, or filled with fresh buffer (or both). In some variations, clean buffer is run from the collection layer to the detector layer until glucose is not detected, and then valves between the waste reservoir and the detector layer are shut to prevent later contamination. Valves between the detector layer and the collection layer may also be shut. The collection layer may then be primed to collect a new sweat sample.

The steps may be repeated (713) after a predetermined period of time, e.g., less than about 60 minutes, less than about 30 minutes, less than about 20 minutes, less than about 10 minutes, less than about 5 minutes, etc. Similarly, the steps may be repeated for a predetermined period of time, e.g., about 1 hour, about 2 hours, about 3 hours, about 4 hours, about 5 hours, about 6 hours, etc. These periods of time may be set automatically, or may be set manually.

As with the methods described above, these methods may also include the step of inducing a sweat prior to collecting a first sweat sample.

### EXAMPLES

### Example 1

### Investigation of the Effects of a Sweat-Permeable Membrane

A standard pilocarpine iontophoresis was performed simultaneously on the clean dry skin of both arms of a 40 year old male type I diabetic. The skin was wiped after stimulation and a MedOptix (now VivoMedical) Macrovial surface was applied within 1 min following the iontophoresis. The MedOptix Macrovial allows serial samples of sweat to be collected from the same site on the skin. It is made from a plate having a hole therethrough for contact with the skin surface. On the non-skin contacting side of the plate, a capillary tube connects the hole to a collection chamber or vial). A Vaseline-paraffin barrier material (acting as a sweat-permeable membrane) was applied to the site on the right arm before the MedOptix Macrovial was applied. Samples were collected every 10 minutes from the appearance of the first drop of sweat on the end of the MedOptix Macrovial. The subject came in with an initial blood glucose level of about 220 mg/dl, which then stabilized at about 175 mg/dl during the first 40 minutes of sample collection. The subject then drank 10 oz of COKE® producing a rise in blood glucose to about 300 mg/dL.

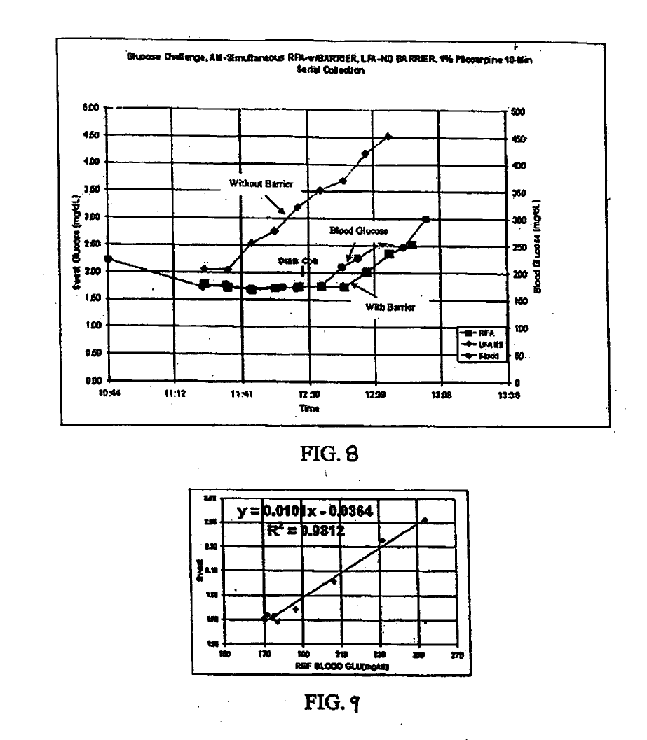

The first two samples from the left arm (having no sweat-permeable membrane), contained approximately 2.0 mg/dl glucose. The glucose concentration of the sweat increased monotonically throughout the rest of the experiment to a maximum of approximately 5.0 mg/dl. This increase in concentration was not correlated to the increase in blood glucose, which began to rise 40 min after the initial rise in glucose in the left arm. In contrast, the glucose samples from the right arm, having the sweat-permeable membrane, remained flat at approximately 1.7 mg/dl and began to rise to a maximum of about 2.5 mg/dl about 10 min after the blood glucose started to rise. These results are shown in FIG. 8.

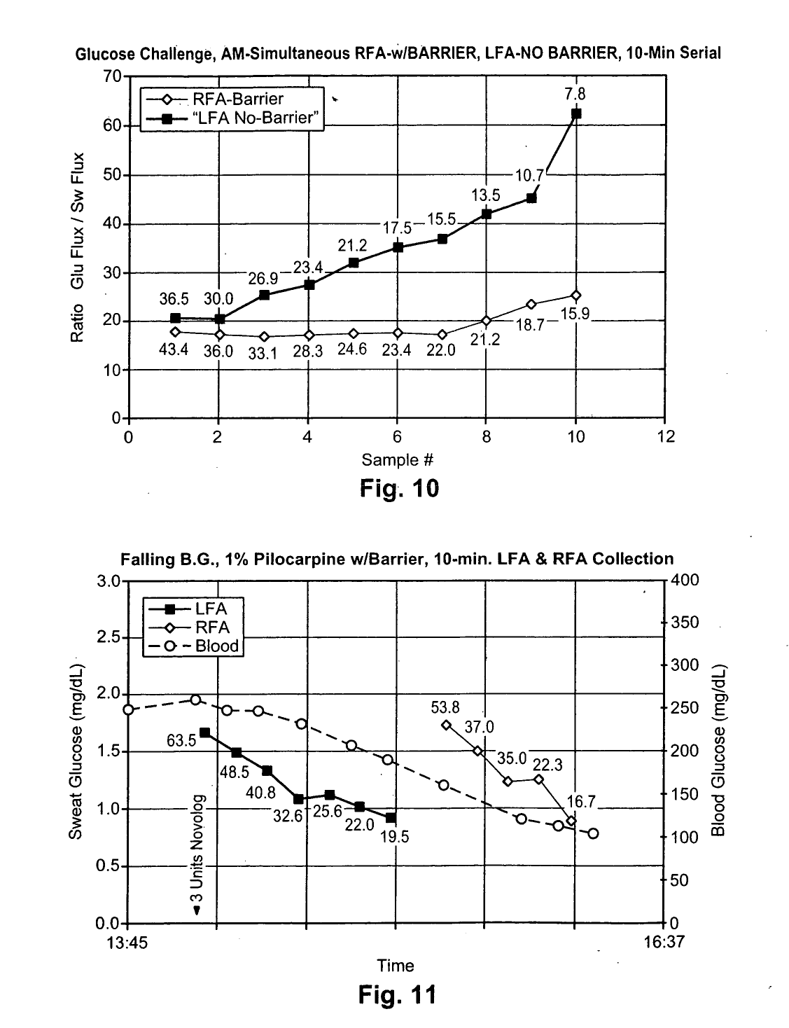

FIG. 9 shows a fit of blood glucose vs. sweat glucose for the site having the sweat-permeable membrane, which has been time-shifted. The blood and sweat glucose values were highly correlated, as shown by the R² of 0.98. The glucose concentration increased throughout the experiment on the site having no sweat-permeable membrane, which is consistent with a source of glucose independent of sweat. FIG. 10 is a plot of the ratio of sweat flux to glucose flux. As shown in that figure, in the case where there is a sweat-permeable membrane, the ratio remains constant while the blood glucose level is constant. Conversely, in the case where there is no sweat-permeable membrane, the ratio increases during this time. Accordingly, this data suggests that the use of a sweat-permeable membrane can act as a barrier to epidermal contaminants and glucose brought to the skin surface via diffusion.

### Example 2

### Correlation of Sweat Glucose to Blood Glucose

Both forearms of the subjects used were wiped with a standard 70% isopropyl alcohol swab. Cotton pads soaked in a buffered saline and 1% pilocarpine solution were applied respectively to the negative and positive electrodes of a standard iontophoresis device. A charge (dose) of 10 mA-min at a current of 1 mA was applied to the electrodes as they were held tightly against the skin of the subjects with elastic straps. The skin was wiped after 10 min of iontophoresis and a MedOptix Macrovial was applied to the site of the positive electrode within 1 min following the iontophoresis. Sample vials were replaced every 10 or 15 min until sweat flow became less than about 10 μl over the collection interval.

Blood glucose levels were determined from capillary finger pricks every 10 minutes using a commercial personal blood glucose meter (ACCU-CHECK ADVANTAGE®, Roche). In some experiments macro-vials were placed simultaneously on the right and left arms, while in others macro-vials were placed first on one arm and then after an hour on the opposite arm. Samples were filtered, diluted and analyzed on a DIONEX® HPAE-PAD system. The protocol varied with the initial state of the subject. For example, if the subject's blood glucose (BG) was high (>200 mg/dL) the subject was asked to follow his normal insulin program to lower BG. Otherwise, the subjects were given a drink containing 35-70 g of glucose at the start of the experiment to produce a rise in BG over the collection period.

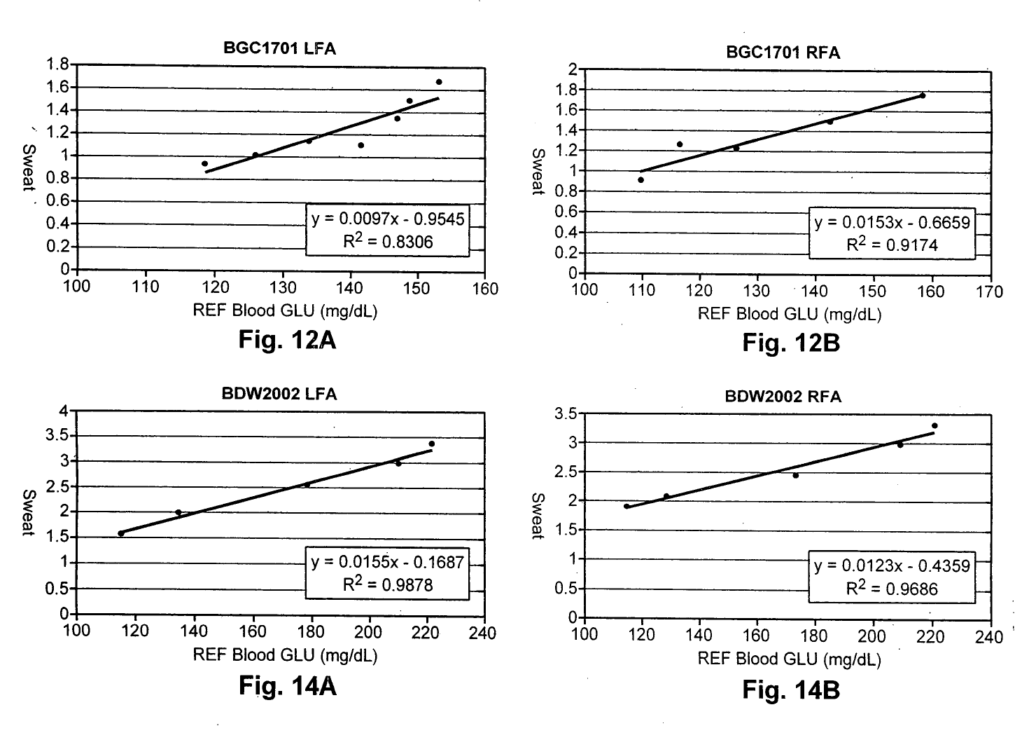

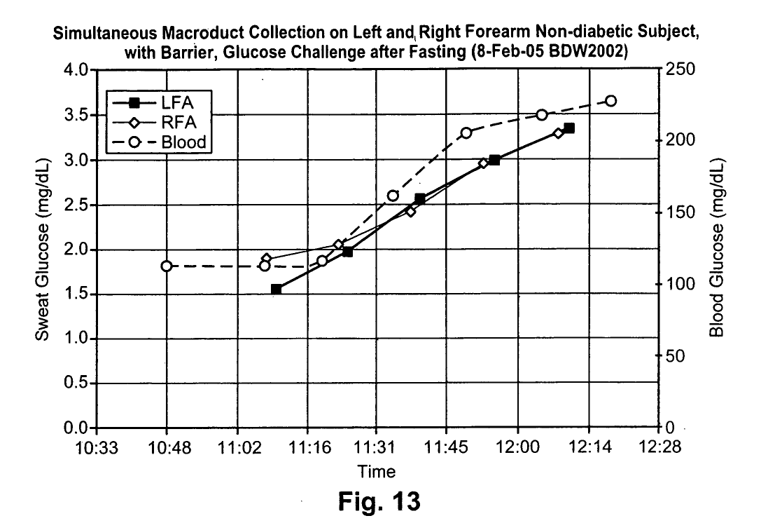

Subject BCG1701, whose results are shown in FIGS. 11 and 12A-B, is a 48 year old female Caucasian, type II diabetic. Subject BDW2002, whose results are shown in FIGS. 13 and 14A-B, is a 39 year old male Asian, non-diabetic.

FIG. 11 shows a typical result for a falling BG. In this experiment the subject arrived with a high (250 mg/dL) BG level. Following the subject's own treatment regime, insulin was injected and samples of sweat and blood were collected from both the left and right forearms. The data shown in FIG. 11 is uncorrected for the offset some subjects demonstrate between their left and right arm. In this figure the BG (circles) decreases from 250 to 100 over the 2.5 hr experiment. The sweat glucose (SG) level is shown for the left forearm (LFA) followed by the right forearm (RFA). The numbers over the SG points give the volume in μl of sweat collected for each sample over the collection interval. FIGS. 12A and 12B show a linear regression plot of interpolated blood glucose vs. sweat glucose for the LFA and RFA respectively. These fits have R² values of 0.83 and 0.92, indicating a high degree of correlation between blood and sweat glucose levels.

FIG. 13 shows experimental results for an experiment with increasing BG. In this experiment the subject was given 75 g of glucose which raised his BG from about 125 to about 200 mg/dL over the course of the experiment. The data plotted in FIG. 13 shows the sweat glucose levels (left axis) of “simultaneous” collections of the LFA and RFA together with the changing blood levels (right axis). FIGS. 14A and 14B show plots of the linear regression of blood vs. sweat glucose for the LFA and RFA. The R² values were 0.99 and 0.97 for LFA and RFA respectively demonstrating a strong correlation between blood and sweat glucose in this experiment.

We claim:

1. A glucose monitoring system comprising:
a patch configured to collect a nanogram quantity of glucose in sweat, wherein the patch comprises a microfluidic collection layer and a detector; and
a measurement device configured to measure the nanogram quantity of glucose.

2. The system of claim 1 wherein the patch further comprises a sweat-permeable membrane configured to act as a barrier to epidermal contaminants and glucose brought to the skin surface via diffusion.

3. The system of claim 2 wherein the sweat-permeable membrane comprises a material that is generally occlusive, but allows sweat to pass therethrough.

4. The system of claim 2 wherein the sweat-permeable membrane comprises a liquid polymer that cures when exposed to oxygen and leaves openings over the sweat gland pores.

5. The system of claim 1 wherein the patch further comprises an adhesive.

6. The system of claim 5 wherein the adhesive is a pressure sensitive adhesive.

7. The system of claim 1 wherein the patch further comprises a mechanism for inducing sweat.

8. The system of claim 7 wherein the mechanism for inducing sweat is mechanical.

9. The system of claim 8 wherein the patch comprises an occlusive backing layer.

10. The system of claim 7 wherein the mechanism for inducing sweat is chemical.

11. The system of claim 10 wherein the patch comprises pilocarpine.

12. The system of claim 11 wherein the patch comprises a penetration enhancer.

13. The system of claim 11 further comprising a mechanism for performing iontophoreris.

14. The system of claim 7 wherein the mechanism for inducing sweat is thermal.

15. The system of claim 14 wherein the patch comprises a heater.

16. The system of claim 1 wherein the microfluidic collection layer comprise a serpentine collection layer.

17. The system of claim 1 wherein the microfluidic collection layer comprises concentric microfluidic channels.

18. The system of claim 7 wherein the mechanism for inducing sweat is in the collection layer.

19. The system of claim 1 further comprising a detector layer, wherein the detector is in the detector layer.

20. The system of claim 19 wherein the detector layer and the collection layer are in fluid communication with each other.

21. The system of claim 20 further comprising a pump.

22. The system of claim 21 wherein the pump is an active pump.

23. The system of claim 21 wherein the pump is a passive pump.

24. The system of claim 21 further comprising a buffer.

25. The system of claim 24 wherein the buffer is at physiological pH and is isotonic.

26. The system of claim 25 wherein the buffer is phosphate buffered saline.

27. The system of claim 1 wherein the detector is an electrochemical detector.

28. The system of claim 27 wherein the detector comprises glucose oxidase.

29. The system of claim 28 wherein the glucose oxidase is in solution.

30. The system of claim 28 wherein the glucose oxidase is substantially immobilized.

31. The system of claim 1 wherein the measurement device comprises a display, a processor, computer executable code for executing a calibration algorithm, and a measurement mechanism for measuring glucose collected in the patch.

32. The system of claim 1 further comprising a device for measuring relative humidity.

33. A patch for use with a glucose monitoring device comprising:
a microfluidic collection layer; and
a detector.

34. The patch of claim 33 wherein further comprising a sweat-permeable membrane configured to act as a barrier to epidermal contaminants and glucose brought to the skin surface via diffusion.

35. The patch of claim 34 wherein the sweat-permeable membrane comprises a material that is generally occlusive, but allows sweat to pass therethrough.

36. The patch of claim 34 wherein the sweat-permeable membrane comprises a liquid polymer that cures when exposed to oxygen and leaves openings over the sweat gland pores.

37. The patch of claim 33 further comprising an adhesive.

38. The patch of claim 33 wherein further comprising a mechanism for inducing sweat.

39. The patch of claim 38 wherein the mechanism for inducing sweat is mechanical.

40. The patch of claim 39 wherein the patch comprises an occlusive backing layer.

41. The patch of claim 38 wherein the mechanism for inducing sweat is chemical.

42. The patch of claim 41 wherein the patch comprises pilocarpine.

43. The patch of claim 42 wherein the patch comprises a penetration enhancer.

44. The patch of claim 42 wherein the patch further comprises a mechanism for performing iontophoresis.

45. The patch of claim 38 wherein the mechanism for inducing sweat is thermal.

46. The patch of claim 45 wherein the patch comprises a heater.

47. The patch of claim 33 wherein the microfluidic collection layer comprises a serpentine collection layer.

48. The patch of claim 33 wherein the microfluidic collection layer comprises concentric microfluidic channels.

49. The patch of claim 38 wherein the mechanism for inducing sweat is in the collection layer.

50. The patch of claim 33 further comprising a detector layer, wherein the detector is in the detector layer.

51. The patch of claim 50 wherein the detector layer and the collection layer are in fluid communication with each other.

52. A method for measuring glucose on the skin surface comprising:
cleaning the skin surface with a glucose solvent;
collecting sweat from the skin surface using a microfluidic collection device; and
measuring the collected glucose.

53. The method of claim 52 further comprising inducing sweat prior to collecting the sweat.

54. The method of claim 53 wherein inducing sweat comprises administering pilocarpine.

55. The method of claim 54 further comprising administering a penetration enhancer.

56. The method of claim 54 further comprising driving the pilocarpine into the skin with iontophoresis.

57. The method of claim 52 wherein measuring comprises measuring nanogram quantities of glucose.

58. A method for measuring glucose on the skin surface comprising:
cleaning the skin surface with a glucose solvent;
collecting sweat from the skin surface in a patch comprising a microfludic collection layer; and
measuring glucose collected in the patch.

59. The method of claim 58 wherein collecting sweat comprises collecting sweat in a microfludic collection layer containing a buffer.

60. The method of claim 58 further comprising pumping a buffer into the microfluidic collection layer.

61. The method of claim 60 wherein pumping the buffer into the microfluidic collection layer is performed after collecting the sweat.

62. The method of claim of claim 58 further comprising repeating the steps of collecting the sweat and measuring the glucose.

63. A method for measuring glucose on the skin surface comprising:
cleaning the skin surface with a glucose solvent;
collecting a first sweat sample from the skin surface in a patch comprising a microfludic collection layer and a detector layer;
transferring the first sweat sample from the collection layer to the detector layer;
measuring glucose in the first sweat sample;
repeating the collection, transferring, and measuring steps at least once.

64. The method of claim 63 wherein collecting the first sweat sample comprises collecting the first sweat sample in a microfludic collection layer containing a buffer.

65. The method of claim 63 wherein the step of transferring the first sweat sample from the collection layer to the detector layer comprises pumping a buffer into the microfluidic collection layer.

66. The method of claim 65 wherein the step of transferring the first sweat sample from the collection layer to the detector layer comprises applying pressure within the microfluidic collection layer.

67. The method of claim 66 wherein the pressure applied is selected from the group consisting of: liquid pressure, gas pressure, and mechanical pressure.

68. The method of claim 66 wherein the pressure applied is pressurized saline.

69. The method of claim 63 wherein the steps are repeated after a predetermined period of time.

70. The method of claim 69 wherein the predetermined period of time is less than about 60 minutes.

71. The method of claim 69 wherein the predetermined period of time is less than about 30 minutes.

72. The method of claim 69 wherein the predetermined period of time is less than about 20 minutes.

73. The method of claim 69 wherein the predetermined period of time is less than about 10 minutes.

74. The method of claim 69 wherein the predetermined period of time is less than about 5 minutes.

75. The method of claim 63 wherein the steps are repeated for a predetermined period of time.

76. The method of claim 75 wherein the steps are repeated for about 1 hour.

77. The method of claim 75 wherein the steps are repeated for about 2 hours.

78. The method of claim 75 wherein the steps are repeated for about 3 hours.

79. The method of claim 75 wherein the steps are repeated for about 4 hours.

80. The method of claim 75 wherein the steps are repeated for about 5 hours.

81. The method of claim 75 wherein the steps are repeated for about 6 hours.

82. The method of claim 63 further including inducing sweat prior to collecting a first sweat sample.

83. The method of claim 82 wherein inducing sweat comprises chemically inducing sweat.

84. The method of claim 82 wherein inducing sweat comprises mechanically inducing sweat.

85. The method of claim 82 wherein inducing sweat comprises thermally inducing sweat.
